(19) United States
(12) Patent Application Publication
(10) Pub. No.: US 2004/0077943 A1
(43) Pub. Date: Apr. 22, 2004

(54) Systems and methods for 3-D data acquisition for microwave imaging

(76) Inventors: Paul M. Meaney, Hanover, NH (US); Keith D. Paulsen, Hanover, NH (US); Margaret W. Fanning, Hanover, NH (US); Timothy Reynolds, Woodstock, VT (US)

Correspondence Address: LATHROP & GAGE LC, 2345 GRAND AVENUE, SUITE 2800, KANSAS CITY, MO, 64108, US

(21) Appl. No.: 10/407,886

(22) Filed: Apr. 4, 2003

Related U.S. Application Data
(60) Provisional application No. 60/370,366, filed on Apr. 5, 2002.

(51) Int. Cl. A61B 5/05

(52) U.S. Cl. 600/430; 600/547

(57) ABSTRACT

Tomographic imaging of biological tissue is achieved through a microwave imaging system and associated methods. An array of antennas are positioned in an illumination tank to surround biological tissue to be imaged. A liquid coupling medium is placed in the illumination tank, and the biological tissue is immersed in the medium. The array of antennas transmit and receive microwave-frequency RF signals that are propagated through the biological tissue. A signal processor is coupled to the antennas to process a demodulated signal representative of the microwave-frequency RF signal received by one or more of the antennas to produce scattered field magnitude and phase signal projections of the biological tissue. These projections may be used to reconstruct a conductivity and permittivity image across an imaged section of the biological tissue to identify the locations of different tissue types (e.g., normal versus malignant or cancerous) within the biological tissue. The liquid coupling medium may include glycerol in a solution with water or saline, or in an emulsion with water, and oil and an emulsifier. The liquid coupling medium containing glycerol provides a low-contrast medium that is beneficial when imaging low-permittivity objects, such as human breast tissue.

### RELATED APPLICATIONS

This application claims priority to U.S. provisional application serial No. 60/370,366, filed Apr. 5, 2002, entitled “OPTIMAL COUPLING LIQUID FOR MICROWAVE TOMOGRAPHIC BREAST IMAGING” and which is incorporated herein by reference.

### BACKGROUND

Numerous techniques exist for determining the makeup or condition of in vivo tissue of a human being or other animal, such as traditional X-rays, X-ray computed tomography (CT scan) and Magnetic Resonance Imaging (MRI). Specifically with respect to the human breast, X-ray mammography is a diagnostic technique frequently used for detecting cancer cells and other tissue abnormalities in females. Frequent screenings through mammograms increase the chances of detecting cancer cells at early stages of development, which consequently makes the cancer easier to treat and increases the survival rate and quality of life for the female.

Despite the advantages of X-ray mammography, this technique has certain drawbacks. For example, X-ray mammography has a somewhat low sensitivity for detecting cancer cells, especially in cases of women with radiographically dense breasts, and frequently identifies false positive signals of cancerous or abnormal tissue.

As an alternative to traditional diagnostic techniques, it has been suggested to use microwave illumination to image biological tissue for cancer screening. This type of microwave imaging measures the alteration of a microwave signal propagated through biological tissue at a certain frequency and reconstructs an electrical property image of the tissue.

The human breast is a good candidate for diagnostic microwave imaging because of the often high contrast in electrical properties between normal and malignant breast tissue. Healthy breast tissue has a low permittivity (εᵣ) relative to malignant tumors, which have electrical properties more akin to biological saline due to the rapid metabolism of cancerous cells and associated angiogenesis. This difference in permittivity generates a contrast between normal and malignant breast tissue that is considerably higher than for other anatomical sites. Some examples of microwave imaging techniques that have been suggested for imaging biological tissue include confocal imaging and tomographic techniques.

Microwave imaging, as a diagnostic technique for in vivo tissue, however, faces a number of obstacles. Full 3-dimensional (3-D) microwave tomographic imaging requires, for example, a Gauss-Newton iterative image reconstruction scheme, and a matching of a numerical model to the actual physics of an imaging apparatus. Problems that typically arise with this scheme include the high cost and long data acquisition times associated with collecting the necessary measurements for microwave tomographic imaging. Other problems arise because of inaccuracies of the image reconstruction algorithm being implemented.

Another obstacle facing in vivo tissue microwave imaging—specifically human breast tissue imaging—is the low permittivity of such tissue. For diagnostic microwave imaging, a coupling medium, typically a liquid, must be used between the tissue and microwave antennas. Saline solutions have been used as a coupling medium because of the low contrast of the solution with the high water content of typical body tissue, and the low cost and suitability of the solution for human contact. However, healthy breast tissue has a lower permittivity than most other human tissues because of the high percentage of fat content, and as such, saline and other solutions do not have a sufficiently low permittivity as to create suitably low contrast with healthy breast tissue, resulting in poor microwave imaging quality. Various alcohols have low relative permittivity values, which range roughly from 15 to 30 at 900 MHz, depending on the length of the carbon chain. Of this family, ethyl alcohol has the highest permittivity and is most soluble in water. However, large amounts of ethyl alcohol must be added to water to bring the mixture down to a relatively low permittivity value, and the level of fumes given off by such a mixture would be dangerous in a clinical situation.

The conductivity (σ) of the coupling medium is also of concern. While a low permittivity liquid is desired, if the value is too low, the conductivity of the coupling medium cannot be independently controlled through the addition of sodium chloride (NaCl). A certain level of conductivity is necessary for microwave antennas to function properly. Other parameters that adversely affect microwave imaging of breast tissue when saline is used as a coupling medium include: (a) excessive wrapping of the measured electrical field phase values, (b) unwanted artifacts that arise due to 3D propagation when image reconstruction assumes a 2-dimensional (2-D) model, and (c) imbalances in the real and imaginary components of the complex reconstruction parameter, k², which represents the wave number squared.

Some early examples of tomographic imaging systems include a system developed by Jofre et al and improved upon by Broquetas et al, which utilized an array of 64 horn antennas operating at 2.45 GHz in water as a coupling medium. Another system developed by Semenov et al utilized a tomographic instrument with a moving transmitter and receiver array submerged in a saline coupling medium for acquiring data on phantoms, animals and human patients. In the Semenov et al system, varying concentrations of sodium chloride in water were used depending on the imaging object size for recovering 2-D and 3-D images. However, these imaging systems suffer from having high contrast between the coupling medium and certain biological tissues, especially human breast tissue.

Biological tissue microwave imaging, as of yet, has not been widely utilized because of the difficulties in efficient data collection, poor reconstruction algorithms, and, with respect to in vivo breast tissue, lack of a suitable coupling medium with the necessary physical properties.

### SUMMARY

A microwave imaging system and associated methods are provided for tomographic imaging of biological tissue. In one aspect, the system includes an illumination tank into which biological tissue is placed, an array of antennas extending into the tank for transmitting and receiving microwave-frequency RF signals, and a signal processor coupled to the antennas. The signal processor is configured for processing the microwave-frequency RF signals propagated through the biological tissue to produce scattered magnitude and phase signal projections. These projections may be used to reconstruct conductivity and permittivity plots across an imaged section of the biological tissue to identify the locations of different tissue types (e.g., normal versus malignant or cancerous) within the biological tissue. The biological tissue may include, for example, human in vivo tissue, such as breast tissue.

In another aspect, the array of antennas extend through seals positioned in bores formed in an illumination tank base. The array of antennas generally surround the biological tissue being imaged, and the portion of the antennas extending outside the illumination tank may be mounted onto a mounting platform. The seals allow the antennas to be moved vertically within the illumination tank by an actuator moving the mounting platform such that data collection for image reconstruction may take place at a number of transverse cross-sectional locations through the biological tissue without a liquid coupling medium within the tank leaking out of the tank.

Alternatively, a drive shaft of the actuator extends through one or more seals positioned in one of the illumination tank base bores. Thus, the array of antennas are fully positioned, along with the mounting platform, within the illumination tank. A coaxial connector bulkhead feed through adapter may be provided for positioning in a bore formed in a illumination tank sidewall so that communications cabling may be extended from the antennas to system electronics (i.e., signal processor and other electronics) disposed outside of the tank.

An optical scanner may be used to optically scan the biological tissue placed within the illumination tank. The reconstructed microwave images formed from the signals received by the antennas and processed by the signal processor may then be spatially co-registered with a 3-D rendering of the outer surface of the biological tissue.

In another aspect, the array of antennas are monopole antennas each formed of a rigid coaxial cable. The antennas have a base region and a tip region, with the tip region having an outer conductor of the coaxial cable removed. The tip region transmits and receives microwave-frequency RF signals, and the base region serves as a transmission line to carry signals from the tip region to a connector that interfaces with communications cables extending to the system electronics.

In another aspect, the array of antennas may be waveguide antennas, or any other form of antenna. The antennas are each mounted with a support rod that extends through one or more seals positioned in a bore formed in the illumination tank base.

In yet another aspect, two independently controlled arrays of antennas facilitate 3-dimensional microwave signal interrogation of the biological tissue. Each antenna array may be moved to a particular vertical position such that microwave-frequency RF signals transmitted by an antenna of one array, and received by an antenna of another array, travel out of a transverse imaging plane with a diagonal component, facilitating true 3-D data collection.

A low-contrast liquid coupling medium is also disclosed to facilitate the transmission of microwave-frequency RF signals from the array of antennas to the biological tissue and back to the antennas. In one aspect, the liquid coupling medium is formed of a solution of water, or saline, with glycerol (also referred to herein as “glycerine”) to provide ideal electrical properties when imaging low permittivity tissue, such as human breast tissue. The volumetric ratios of glycerol and water in the liquid coupling medium may be optimized for good contrast depending on the particular permittivity values of the tissue being imaged. For example, volumetric ratios of between about 50:50 and 90:10 glycerol to water have been found to work well when imaging human breast tissue. Glycerol provides the advantages of essentially not being harmful when contacting human skin, being environmentally friendly, being readily soluble in water and relatively inexpensive.

### BRIEF DESCRIPTION OF THE DRAWINGS

FIG. 1 is a block diagram of one microwave imaging system;

FIG. 2 is a perspective view of an illumination tank assembly of the microwave imaging system;

FIG. 3 is a side elevational view of the illumination tank assembly of FIG. 2;

FIG. 4 is a schematic diagram of an array of antennas and straight line signal projections propagated through an object having regions of differing conductivity and permittivity;

FIG. 5A is a top plan view of a monopole antenna; FIG. 5B is a side elevational view of the monopole antenna;

FIG. 6 is a close-up view of the illumination tank assembly of FIG. 2 showing an antenna, seals, mounting platform, and antenna connector;

FIG. 7 is a perspective view of another illumination tank assembly utilizing monopole antennas;

FIG. 8 is a side elevational view of the illumination tank assembly of FIG. 7;

FIG. 9 is a partial sectional view of another illumination tank assembly utilizing waveguide antennas;

FIG. 10 is a perspective view of another illumination tank assembly with first and second arrays of antennas;

FIG. 11 is a perspective view of the illumination tank assembly of FIG. 10 showing the first and second arrays of antennas inside an illumination tank;

FIG. 12 is a schematic diagram of the illumination tank assembly of FIG. 10 showing application of the system in imaging in vivo tissue of a person;

FIG. 13 is a conceptual diagram of the slice averaging width of an object being imaged;

FIG. 14 shows a plot of the averaged diameter of the object of FIG. 13 as a function of the averaging width center with respect to the center of the object.

FIG. 15 shows plots of the relative permittivity versus frequency for a range of mixture ratios of glycerol and water;

FIG. 16 shows plots of the conductivity versus frequency for a range of mixture ratios of glycerol and water;

FIG. 17 shows plots of the unwrapped electric field scattered phases for a range of frequencies measured at a number of antenna receivers due to microwave signals propagated through a human breast pendant in a 70:30 (X_{Glyc}=70%) glycerol/water liquid coupling medium;

FIGS. 18A and 18B show reconstructed images of low-dielectric agar gel cylinders with and without inclusions from microwave signal measurement data at an operating frequency of 900 MHz; the cylinders positioned in 0.9% saline, and X_{Glyc}=50% and X_{Glyc}=60% glycerol/water and the data measured at a number of antenna receivers;

FIGS. 19A and 19B show reconstructed images of low-dielectric molasses cylinders with and without inclusions from microwave signal measurement data at an operating frequency of 900 MHz; the cylinders positioned in 0.9% saline, and X_{Glyc}=50% and X_{Glyc}=60% glycerol/water and the data measured at a number of antenna receivers; and

FIGS. 20A-20C show reconstructed images in three planes of a human breast pendant in the liquid coupling medium formed of a X_{Glyc}=70% glycerol/water with microwave signals transmitted at 500 MHz, 700 MHz and 1000 MHz and surrounded by the array of monopole antennas.

### DETAILED DESCRIPTION OF ILLUSTRATED EMBODIMENTS

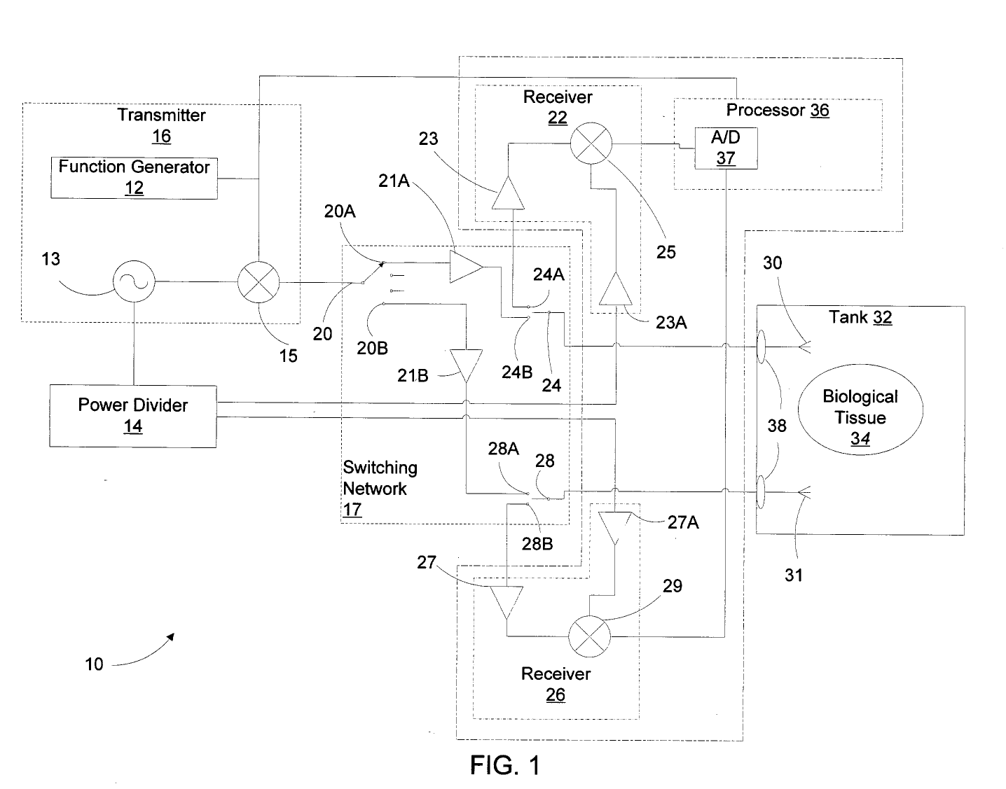

FIG. 1 shows a block diagram of a microwave imaging system 10 in accord with one embodiment. System 10 is configured for examining biological tissue 34 in illumination tank 32. In one embodiment, system 10 is particularly useful in determining whether biological tissue 34 contains sections of abnormal tissue, such as malignant or cancerous tissue.

System 10 includes receivers 22 and 26. Receivers 22, 26 represent a plurality of receivers configured for receiving RF signals from antennas 30 and 31, respectively; these RF signals may be microwave signals ranging in frequency from 300 MHZ to 3 GHz. Antennas 30, 31 may be more than two antennas to form an array of antennas, and may be coupled through seals 38 (e.g., hydraulic seals) in illumination tank 32 to receive microwave signals. Each of antennas 30 and 31 has a respectively coupled receiver (e.g., receivers 22 and 26) configured for receiving and for demodulating the signals. The demodulated signals may be IF (intermediate frequency) signals ranging in frequency from 1 KHz to 1 MHz, but may include other frequencies as a matter of design choice.

System 10 also includes processor 36 coupled to receivers 22, 26 and configured for processing a digital representation of the demodulated signals. In one embodiment, the processor includes an analog to digital (A/D) converter 37 which digitizes the demodulated signals from each of the receivers. Processor 36 then processes the digitized signals to determine phase differences between digital representations of the modulating waveform of the transmitted signal and the demodulated signals. As used herein, the transmitted signal includes a carrier signal modulated by a modulating signal.

To illustrate, if four receivers receive signals from their respectively coupled antennas, then each of the four receivers demodulates a received signal from their respective antenna to extract a demodulated signal. A/D converter 37 digitizes each of the four demodulated signals and processor 36 processes those four demodulated signals by comparing each demodulated signal to a digital representation of the modulating waveform of the transmitted signal.

In one embodiment, the combination of any number of receivers 22, 26 and processor 36 may be referred to as signal processor 39. Signal processor 39 examines the phase differences between a particular received demodulated signal and the modulating waveform of the transmitted signal to produce scattered magnitude and phase signal projections due to the presence of biological tissue 34. The projections may be used to reconstruct electrical property images for use in identifying tissue types, such as healthy tissue versus malignant or cancerous tissue. For example, signal processor 39 can be configured to implement a log-magnitude/phase Gauss-Newton reconstruction algorithm as described in “Microwave image reconstruction utilizing log-magnitude and unwrapped phase to improve high-contrast object recovery” by P. M. Meaney, K. D. Paulsen, B. W. Pogue, and M. I. Miga, IEEE Trans. MI, Volume MI-20, 104-116 (2001), incorporated herein by reference. The scattered signals from each of the antennas configured to receive the signals may then be used in constructing a conductivity and permittivity image of biological tissue 34 under examination. The term “scattered” refers to the difference in phase and magnitude between imaging situations where biological tissue 34 is present in tank 32 and when such tissue is not present. The differences may be computed in log format for the magnitude and phase angle for the phase.

In one embodiment, processor 36 digitally low pass filters the signal from AID converter 37 such that processor 36 may examine a frequency-isolated (e.g., filtered) version of the demodulated signal. In other embodiments, an analog Low Pass Filter (LPF) is coupled between receivers 22, 26 and A/D converter 37 to perform similar functionality, as is known in the art. While this illustration describes system 10 with four receivers and four antennas, the embodiment is not intended to be limited to the number of receivers and antennas of the illustration; nor is the embodiment intended to be limited to the number of receivers and antennas shown in FIG. 1.

Each of receivers 22 and 26, in one embodiment, includes two amplifiers (e.g., 23, 23A, 27 and 27A, respectively) and a signal multiplier (e.g., 25 and 29, respectively). Amplifiers 23, 27 are configured for amplifying RF signals received from antennas 30, 31; amplifiers 23A and 27A are configured for amplifying the reference carrier signal from a power divider 14. Once amplified, signal multipliers 25, 29 demodulate their respectively received signals by multiplying the signals with the amplified carrier signal.

System 10 includes, in one embodiment, transmitter 16 configured for generating the transmitted signal constructed from the carrier signal and the modulating waveform. Transmitter 16 includes RF signal generator 13 configured for generating the carrier signal. Transmitter 16 also includes signal multiplier 15 and function generator 12. Function generator 12 is coupled to signal multiplier 15, as is RF signal generator 13. Function generator 12 is configured for generating a modulating waveform used to modulate the carrier signal as applied by signal multiplier 15. In another embodiment, function generator 12 is coupled to processor 36 for comparison of the original modulating waveform to that of the extracted demodulated signal. It should be further noted that the transmitter 16 and the associated components may be consolidated into a single transmitter unit, such as Agilent model ESG4432 Signal Generator, to provide the RF, carrier signal and the modulating waveform.

In one embodiment, system 10 includes power divider 14 configured for receiving the carrier signal from RF signal generator 13. Power divider 14 splits the carrier signal into multiple same signals, typically of lesser magnitude or gain. These signals are applied to signal multipliers 25 and 29 through associated amplifiers 23A and 27A such that receivers 22 and 26 may demodulate received signals according to well-known trigonometric equations.

A switching network 17 is configured for applying the transmitted signal to one or more of antennas 30, 31, in one embodiment. For example, switching network 17 may apply the transmitted signal to antenna 30 such that the transmitted signal passes through biological tissue 34. Switching network 17, during transmission of the signal via antenna 30, is also configured to receive the transmitted signal via antenna 31 through a switch selection.

Switching network 17 includes an N-connection switch 20 having an RF input terminal coupled to an output of signal multiplier 15. N-connection switch 20 also has “N” number of RF output terminals selectively coupling antennas 30 and 31 to signal multiplier 15, where N is an integer greater than 1. Switching network 17 also includes transmit/receive switches 24 and 28 respectively coupled for selectively switching between either a receive mode or a transmit mode of antennas 30 and 31. To illustrate, as N-connection switch 20 is selected (e.g., closed) at node 20A to transmit the signal from signal multiplier 15 amplified by amplifier 21A, transmit/receive switch 24 is selected (e.g., “closed”) to node 24B for conducting the signal to antenna 30. Accordingly, N-connection switch 20 is “open” at node 20B. Transmit/receive switch 28 is selected (e.g., closed) at node 28B to receive, via antenna 31, the signal transmitted through antenna 30. While this embodiment illustrates one manner in which an antenna transmits one signal and other antennas receive the transmitted signal by means of switching network 17, this embodiment is not intended to be limited to the selection of transmit and receive antennas described herein. For example, multiple transmitters, each generating a transmitted signal with a unique carrier frequency, may be employed such that switching network 17 selectively transmits through a plurality of antennas and selectively receives through a plurality of antennas.

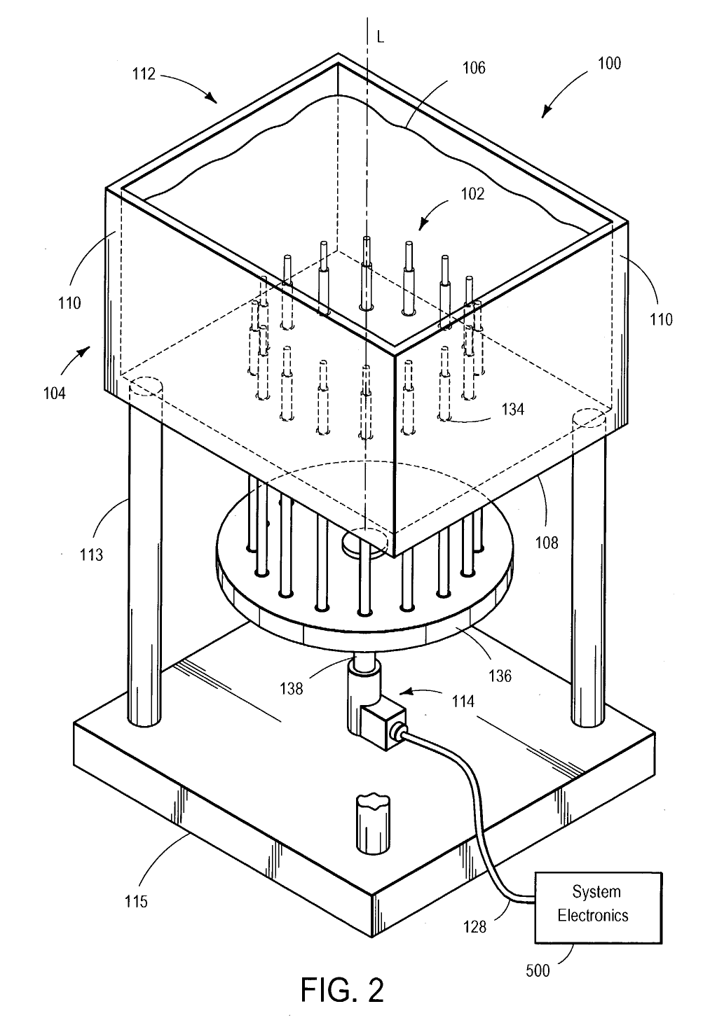

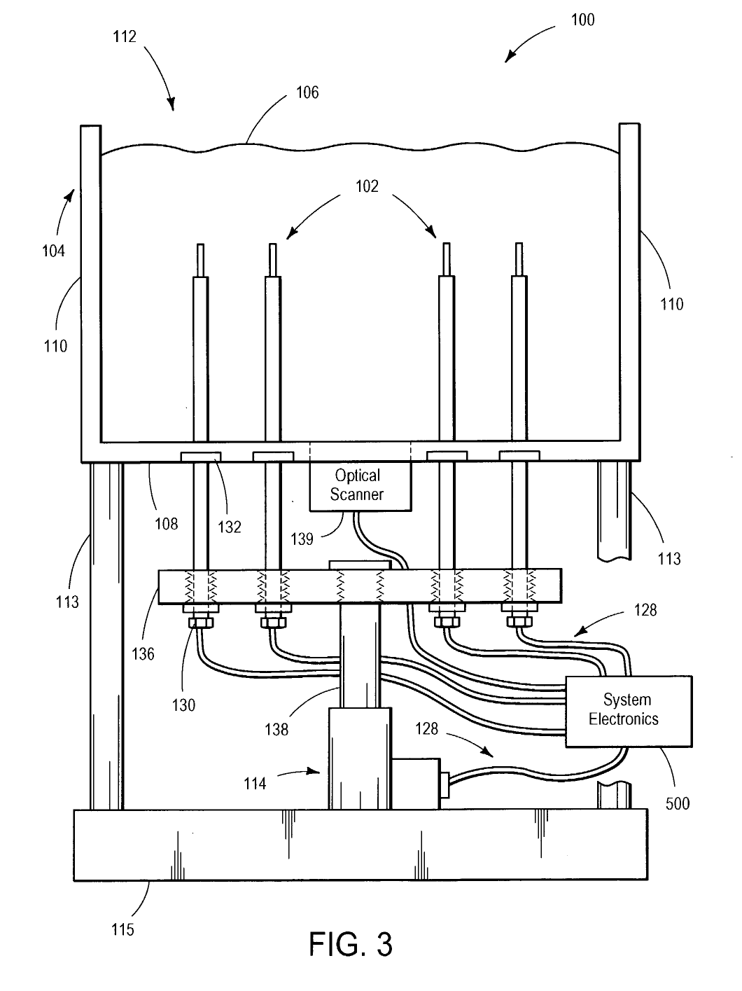

FIGS. 2 and 3 show one illumination tank assembly 100 for microwave illumination of biological tissue. An array of antennas 102 are positioned to extend into an illumination tank 104 holding a volume of a liquid coupling medium 106. Liquid coupling medium 106 facilitates the transmission of microwave-frequency RF signals from the antennas 102 to and through biological tissue (e.g., biological tissue 34, FIG. 1) and back to antennas 102. The specific physical properties of liquid coupling medium 106 will be discussed more fully herein. Illumination tank 104 may have a base 108 and one or more sidewalls 110 depending on the shape of the tank (e.g., one sidewall if the tank is cylindrical in shape, multiple sidewalls if another shape). The array of antennas 102 preferably surround biological tissue (e.g., tissue 34, FIG. 1, as human in vivo tissue, such as breast tissue) that is extended into liquid coupling medium 106 through an open end 112 of illumination tank 104 and radiate microwave-frequency RF signals through the tissue. In one embodiment, array of antennas 102 includes 16 individual antennas; however, any number of antennas may be used depending on the desired amount of imaging detail. In FIG. 3, only 4 antennas are depicted for clarity of assembly 100 and the components thereof. An actuator 114, for example a computer-controlled linear actuator, may be provided to drive the movement of the array of antennas 102 vertically along a longitudinal axis L of illumination tank 104 such that microwave-frequency RF signals may be transmitted and received by antennas 102 at varying transverse, or horizontal, imaging planes orthogonal to the longitudinal axis L and through the biological tissue. Actuator 114, and other components of assembly 100, including illumination tank 104, may be supported by a base support 115; a series of legs 113 may extend downward from illumination tank 104 to the base to support tank 104. Base support 115 may be provided with wheels (not shown) such that at least a portion of assembly 100 supported by base support 115 is portable and may be easily moved across a surface.

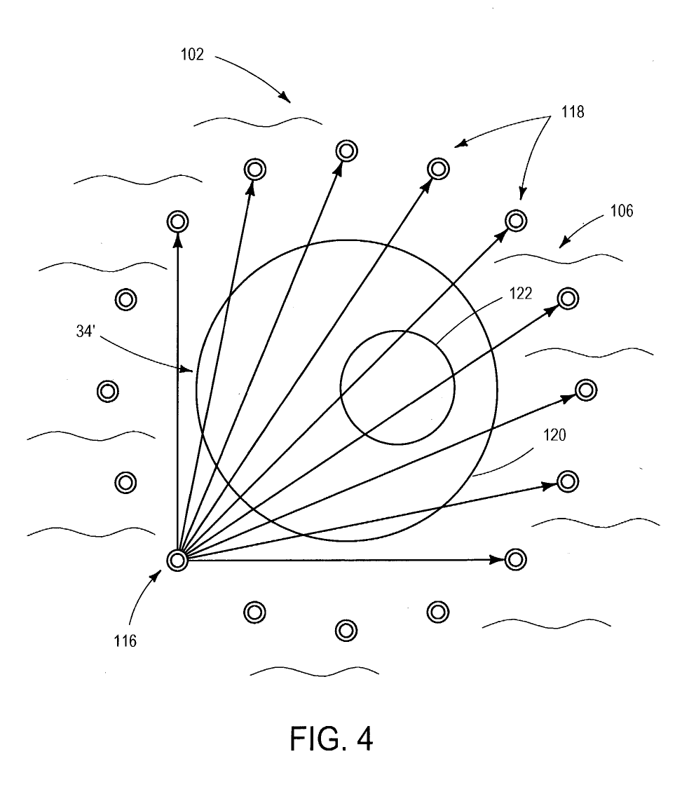

FIG. 4 shows one antenna 116 of the array of antennas 102 transmitting microwave-frequency RF signals that are received by other antennas 118 of the array of antennas 102. A portion of these transmitted signals are propagated through a first portion 120 of biological tissue 34′ (e.g., biological tissue 34, FIG. 1), and another portion of such signals are propagated through both first portion 120 and a second portion 122 of biological tissue 34′, each portion having a unique set of conductivity and permittivity characteristics. It should be noted that the biological tissue 34′ and antenna array 102 are submerged in liquid coupling medium 106 in illumination tank 104. It is the varying conductivity and permittivity characteristics, or electrical properties, of the first and second portions 120, 122 that may be mapped for each chosen transverse imaging plane through biological tissue 34′. This mapping shows where non-uniform regions exists in biological tissue 34′ which may correspond to tissue abnormalities, such as malignancy. For example, when imaging breast tissue, first portion 120 may correspond to healthy tissue and second portion 122 may correspond to otherwise abnormal and/or malignant tissue.

System electronics 500 (i.e., transmitter 16, power divider 14, switching network 17, receivers 22, 26 and processor 36, FIG. 1) provide control over the operation of actuator 114 and the generation and reception of microwave signals through the array of antennas 102. As shown in FIG. 3, coupling of system electronics 500 to the array of antennas 102 and actuator 114 may be through communication cables 128 (e.g., coaxial electrical cables, fiber optic cables or digital electronic ribbon cables) as a matter of design choice. Communication cables 128 coupling the array of antennas 102 and system electronics 500 are omitted from FIG. 2 for clarity. Each antenna 102 has a connector 130 formed therewith to which one communication cable 128 is attached. Upon generation of a microwave-frequency RF signal by system electronics 500, such signal is carried by one or more of communication cables 128 to the respective antenna 102 for transmission. As with system 10 of FIG. 1, the microwave-frequency RF signals are, for example, signals ranging in frequency from 300 MHz to 3 GHz. Other frequencies may also be used as a matter of design choice depending on the electrical properties of liquid coupling medium 106 and biological tissue 34′. Since the electrical properties of the various portions of biological tissue 34′ vary depending on the frequency of microwave transmission, a more complete mapping of non-uniform regions in biological tissue 34′ may be realized by imaging at a number of transmission frequencies. Transmitting antenna 116 of the array of antennas 102 then transmits the microwave signal through biological tissue 34′, as shown in FIG. 4. Receiving antennas 118 then detect the microwave signals propagated through biological tissue 34′, and send the detected signals through the respective communication cables 128 back to system electronics 500. Each antenna that may act as a receiving antenna 118 has a receiver (e.g., receivers 22, 26, FIG. 1) associated therewith. System electronics 500 may then store the signal information received and reconstruct maps of the conductivity and permittivity characteristics of biological tissue 34′. The array of antennas 102 may all be positioned in the same transverse plane through biological tissue 34′ so that the conductivity and permittivity characteristics of biological tissue 34′ (representative of signals that traveled in the transverse plane from transmitting antenna 116 to receiving antenna 118) may be mapped at specific vertical elevations of biological tissue 34′.

Once data acquisition is completed at a specified transverse plane through biological tissue 34′, actuator 114 may move the array of antennas 102 vertically up or down to select imaging at another vertical elevation of biological tissue 34′ (i.e., another transverse plane). The vertical movement of the array of antennas 102 with actuator 114 positioned outside of illumination tank 104 is facilitated by extending antennas 102 through a series of seals 132 (e.g., Teflon hydraulic seals) disposed within bores 134 formed into base 108 of illumination tank 104, as shown in FIG. 2. Seals 132 facilitate relatively low-friction translation of antennas 102 while preventing liquid coupling medium 106 from leaking out of illumination tank 104. The array of antennas 102 may be mounted onto a mounting platform 136 that is moved vertically by a drive shaft 138 connected with actuator 114. By the arrangement of assembly 100, system electronics 500 are fully positioned outside of illumination tank 104; this is advantageous because of the vulnerability of electronics to being compromised by liquid coupling medium 106 in tank 104. In another arrangement, an actuator 114 may be provided for each individual antenna of the array of antennas 102 such that each antenna may be positioned vertically and independently.

After a series of digital acquisitions at differing transverse planes through biological tissue 34′ vertically adjusted by actuator 114, biological tissue 34′ may be optically scanned. An optical scanner 139 may be mounted with illumination tank base 108 either within illumination tank 104 or just below tank 104 scanning through an optically clear portion of base 108. The reconstruction of the microwave images, knowing the vertical elevation of each transverse plane with respect to biological tissue 34′, may then be spatially co-registered with a 3-D rendering of the exterior of the biological tissue 34′ (e.g., breast tissue) that has been optically scanned such that non-uniform regions or other abnormalities imaged may be located with a specific visual reference to biological tissue 34′. Alternatively, optical scanning of biological tissue 34′ may take place transversely through optically clear portions of illumination tank sidewalls 110, or the external dimensions of biological tissue 34′ may be determined using ultrasound or mechanical measuring devices without optical scanning.

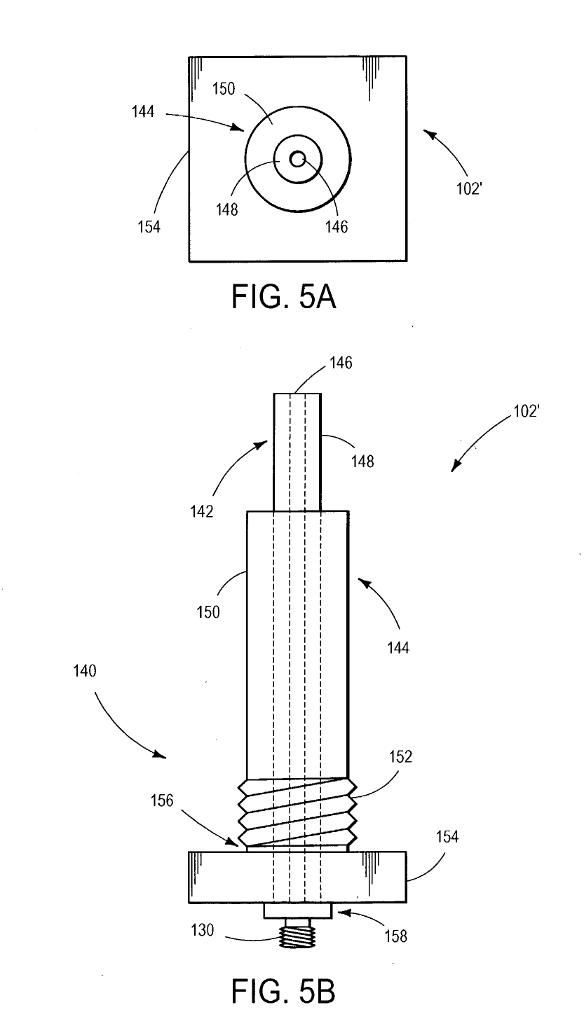

The array of antennas 102 of FIGS. 2 and 3 may be formed as monopole antennas 102′, as shown in FIGS. 5A and 5B. Each monopole antenna 102′ has a base region 140 and a tip region 142 extending therefrom. Base region 140 may be formed of a rigid coaxial cable 144 with a center conductor 146, a cylindrical insulator 148, such as a Teflon insulator, and a rigid, cylindrical outer conductor 150. Base region 140 may also have threads 152 formed onto the outer conductor 150 for securing antenna 102′ into a threaded bore of mounting platform 136, and a mounting flange 154 disposed at a terminating end 156 of the outer conductor 150 to abut mounting platform 136. Tip region 142 is formed of coaxial cable 144 without outer conductor 150, and is the portion of monopole antenna 102′ responsible for direct transmission and reception of microwave-frequency RF signals with liquid coupling medium 106. In this arrangement, base region 140, having center conductor 146 and cylindrical insulator 148 contiguous with tip region 142, acts as a transmission line for signals traveling between tip region 142 and connector 130. Connector 130 may be of any type connector for coupling coaxial cable 144 with communications cable 128, such as a N-connector, SMA, SMB, etc., the particular connector depending on the type of cable 128 (e.g., electrical or fiber optic cable). Connector 130 may also be formed onto a lower end 158 of center conductor 146 and cylindrical insulator 148 below mounting flange 154.

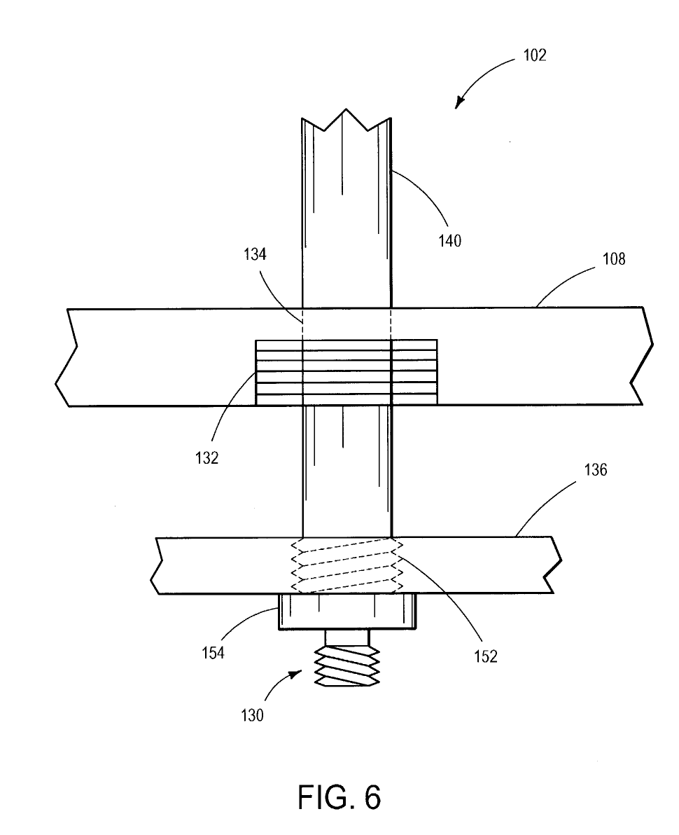

FIG. 6 shows the details of one of the array of antennas 102 extending through bores 134 of illumination tank base 108 and mounted onto mounting platform 136. Base region 140 of each antenna 102 is surrounded by one or more seals 132 stacked within bore 134 and is shown with threads 152 threadingly received into the threaded bore of mounting platform 136. The number of seals 132 and tolerance with the diameter of base region 140 should be sufficient to withstand the forces induced by the antennas 102 sliding therethrough under the influence of actuator 114 without leakage of liquid coupling medium 106 through seals 132.

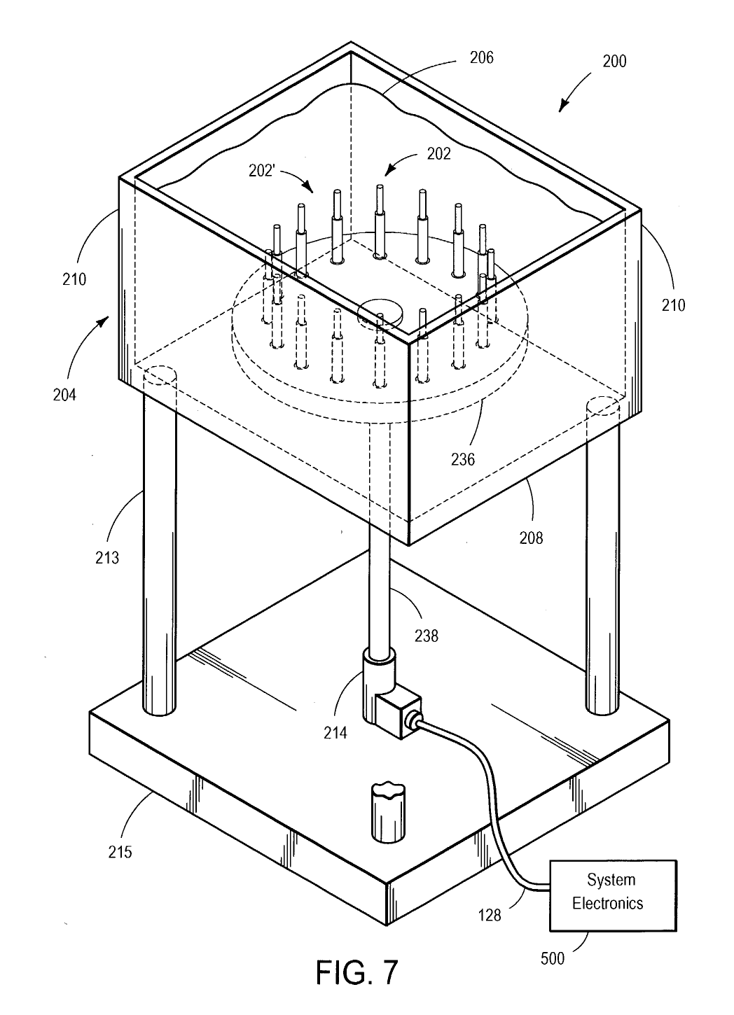

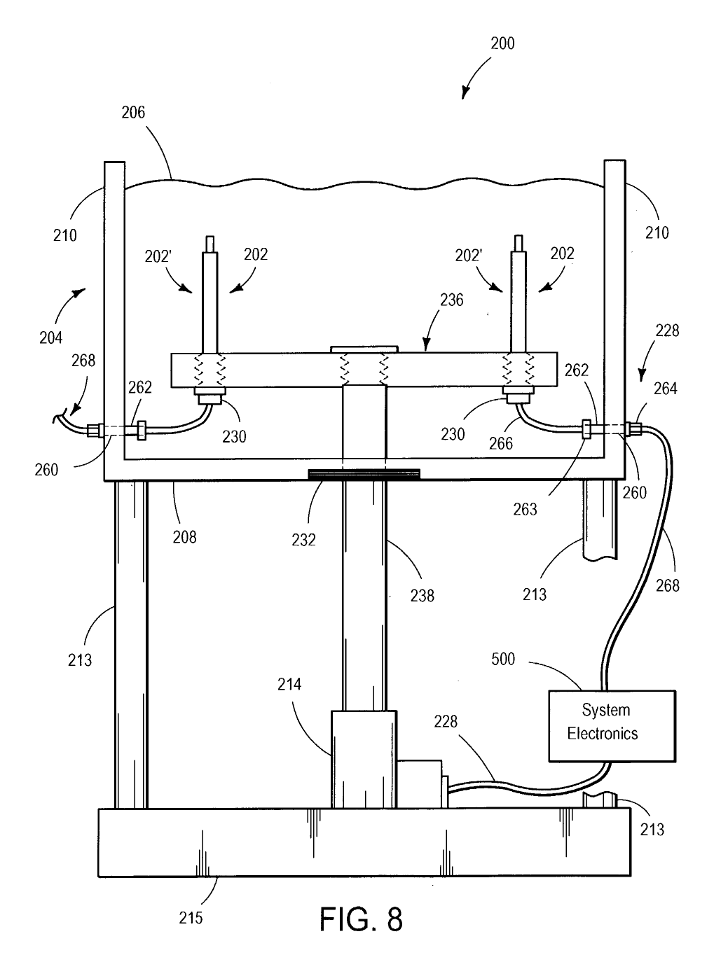

FIGS. 7 and 8 show another illumination tank assembly 200 having similar components to assembly 100 of FIGS. 2 and 3. Assembly 200 has all regions of an array of antennas 202 and a mounting platform 236 disposed within an illumination tank 204. Drive shaft 238 extends through seals 232 into illumination tank 204, as opposed to assembly 100 of FIGS. 2, 3 and 6, where the array of antennas 102 extend through seals 132. The array of antennas 202 may surround biological tissue in the same arrangement as assembly 100 of FIGS. 2 and 3. In FIG. 8, only 2 antennas are depicted for clarity of assembly 200 and the components thereof, but any number of antennas may be implemented (e.g., 16 antennas). Antennas 202 may also be monopole antennas 202′ having the arrangement shown for monopole antenna 102′ of FIGS. 5A and 5B. Communications cables 228, connected with a connector 230 of each antenna 202, may be routed through a liquid coupling medium 206 upward and out of illumination tank 204 over a sidewall 210 to system electronics 500. Communications cables 228 extending from antennas 202 to system electronics 500 are omitted from FIG. 7 for clarity. Alternatively, bores 260 may be extended through any of the illumination tank sidewalls 210 or base 208 such that each communication cable 228 may exit illumination tank 204 proximal to illumination tank base 208 to communicatively couple antennas 202 with system electronics 500. In this arrangement, communications cables 228 are less likely to interfere with any biological tissue placed in illumination tank 204. One configuration for preventing liquid coupling medium 206 from leaking out of illumination tank 204 through the tolerance space between communications cables 228 and associated bores 260 is to use a coaxial bulkhead feed through adapter 262. Bulkhead adapter 262 may be, for example, a female-to-female SMA type adapter, with male connectors 263, 264 (e.g., SMA type connectors) secured to opposing ends thereof. Bulkhead adapter 262 thus facilitates improved communications cable management in assembly 200 by positioning cables so as to provide minimal spatial interference with operation of the system. A first communications cable section 266 may be attached to connector 230 on one end and to bulkhead adapter 262 via male connector 263 disposed within illumination tank 204 on the opposing end, and a second communications cable section 268 may be attached to bulkhead adapter 262 via male connector 264 disposed outside of tank 204 on one end, and to the system electronics 500 on the opposing end. Enough length of first communications cable section 266 should be provided to allow for a range of vertical movements of the attached array of antennas 202 by actuator 214. Similar to assembly 100 of FIGS. 2 and 3, actuator 214, and other components of assembly 200, including illumination tank 204, may be supported by a base support 215, and legs 213 may support tank 204 above base support 215.

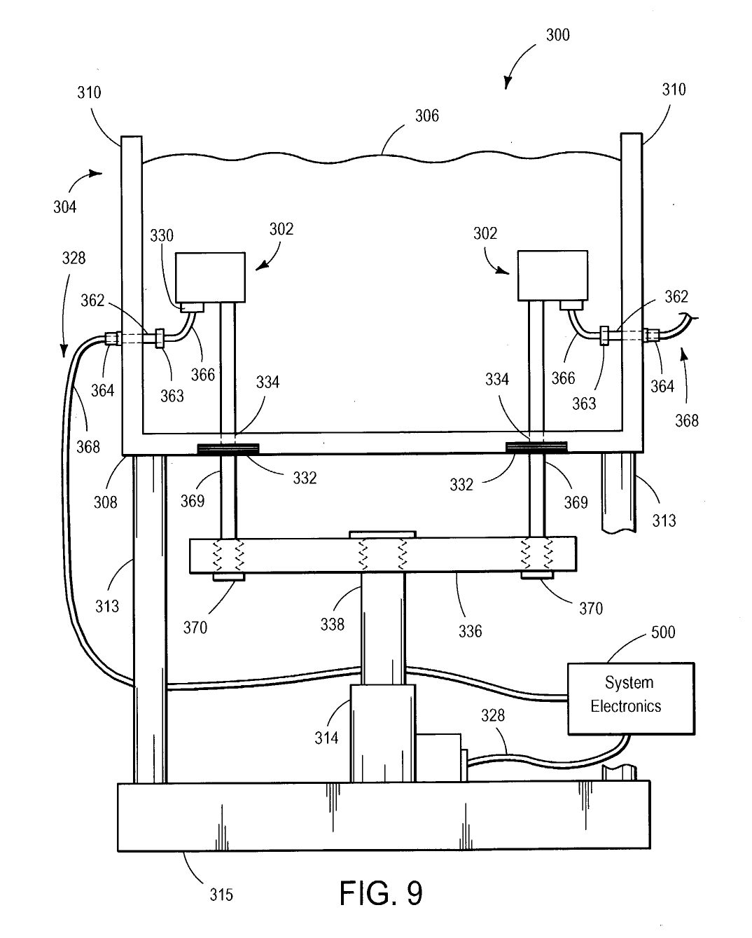

Another microwave imaging assembly 300 is shown in FIG. 9. Assembly 300 is similar to assembly 100 of FIGS. 2 and 3, and assembly 200 of FIGS. 7 and 8, but specifically uses an array of waveguide antennas 302. The array of waveguide antennas 302 may surround biological tissue in the same arrangement as assembly 100 of FIGS. 2 and 3, and assembly 200 of FIGS. 7 and 8. FIG. 9 only shows 2 waveguide antennas for clarity of assembly 300 and the components thereof, but any number of waveguide antennas may be implemented. In assembly 300, the arrangement of an actuator 314, a drive shaft 338 and a mounting platform 336 may be the same as in assembly 100 of FIGS. 2 and 3. Instead of antennas 102 extending through illumination tank base 108 into illumination tank 104, an array of support rods 369 extend through seals 332 disposed within bores 334 formed into an illumination tank base 308. Each support rod 369 has one waveguide antenna 302 mounted therewith on an upper end, and a mounting flange 370 formed at a lower end of the rod 369 to abut mounting platform 336. Support rods 369 may also be threadingly received into threaded bores of mounting platform 336 for mounting thereon.

Similar to assembly 200 of FIGS. 7 and 8, communications cables 328 connected with a connector 330 of each waveguide antenna 302 may be routed through liquid coupling medium 306 upward and out of an illumination tank 304 over one or more sidewalls 310 to system electronics 500. Alternatively, the bulkhead adapter arrangement shown in FIGS. 7 and 8 may be implemented as shown in FIG. 9 to communicatively couple waveguide antennas 302 with system electronics 500. Thus, a first communications cable section 366 may attached to connector 330 on one end and to bulkhead adapter 362 via male connector 363 disposed within illumination tank 304 on the opposing end, and a second communications cable section 368 may be attached to bulkhead adapter 362 via male connector 364 disposed outside of tank 304 on one end, and to system electronics 500 on the opposing end, bulkhead adapter 362 spanning between male connectors 363, 364. Similar to assembly 100 of FIGS. 2 and 3, actuator 314, and other components of assembly 300, including illumination tank 304, may be supported by a base support 315, and legs 313 may support tank 304 above base support 315.

In an alternative arrangement for assembly 300, mounting platform 336 and drive shaft 338 may be positioned in the same configuration as in assembly 200 of FIGS. 7 and 8, with drive shaft 338 extending through seals 332 into illumination tank base 308 through a single bore 334. The array of support rods 369 would then be fully positioned within illumination tank 304.

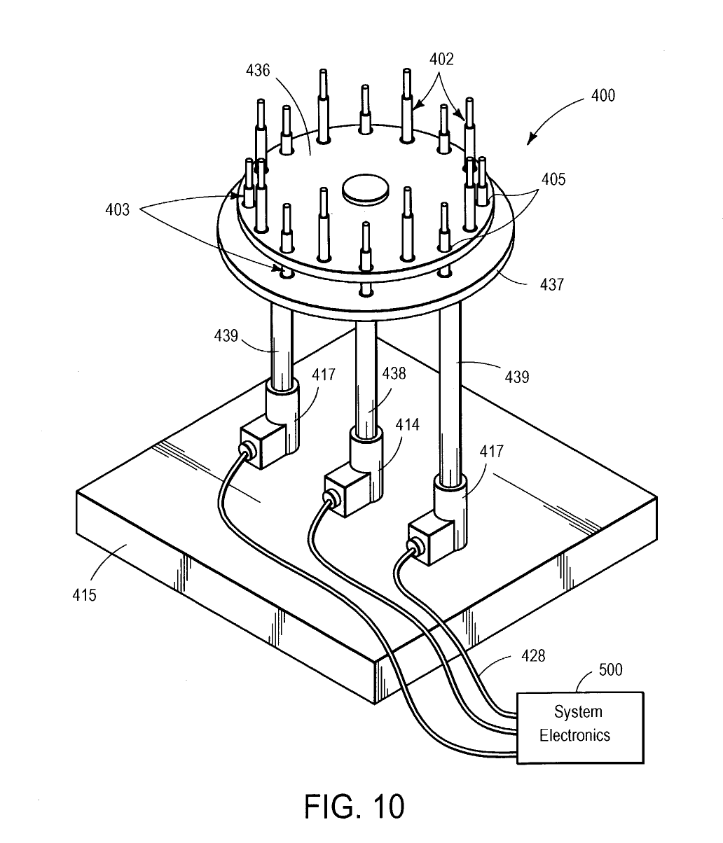

FIGS. 10-12 show another illumination tank assembly 400 utilizing two different, independently controlled arrays of antennas to perform 3-D microwave imaging of biological tissue. This arrangement goes beyond performing data acquisition in a series of transverse slices at various vertical elevations of a biological tissue being imaged, because microwave-frequency RF signals may be transmitted by an antenna array 402 at one vertical elevation, and received by an antenna arrays 403 at another vertical elevation. Thus, microwave-frequency RF signals propagating out of a transverse plane aligned with a transmitting antenna may be detected.

FIG. 10 shows assembly 400 without an illumination tank 404 and communications cables 428 that connected antenna array 402 to system electronics 500, for clarity of the assembly layout. A first array of antennas 402 may be vertically positionable by a first actuator 414 at a first transverse plane P₁, and a second array of antennas 403 may be vertically positionable by one or more second actuators 417 at a second transverse plane P₂. Actuators 414, 417 may be controlled by system electronics 500 coupled therewith by communications cables 428. First and second actuators 414, 417, as well as other components of assembly 400, may also be supported by a base support 415 in a similar fashion to assembly 100 of FIGS. 2 and 3.

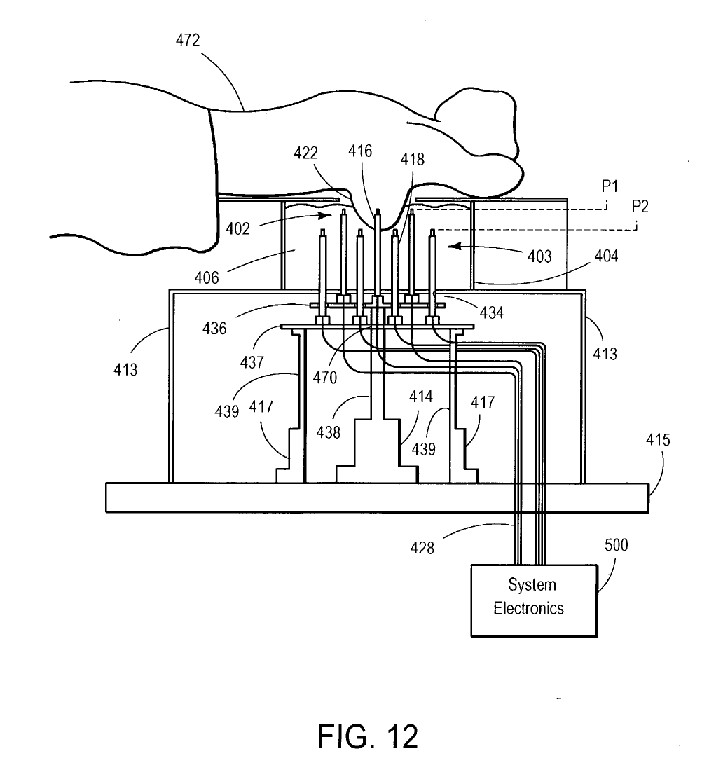

In one embodiment, the arrays of antennas 402, 403 are disposed in an interleaved, circular arrangement with a common diameter. Each array of antennas 402, 403 may include, for example, 8 individual antennas, for a total of 16 antennas between the two antenna arrays; however, the number of antennas used may depend on the desired amount of imaging detail. The antenna arrays 402, 403 may comprise monopole antennas, waveguide antennas, or other antenna types that are compatible with the transmission and reception of microwave signals. By positioning a transmitting antenna 416 of one antenna array (e.g., first array 402) at a different vertical elevation with respect to biological tissue 422 than one or more receiving antennas 418 of the other array (e.g., second array 403), as shown in FIG. 12, data acquisition may take place for out-of-plane propagation. The vertical distance between the antenna arrays 402, 403, and the particular location of transmitting antenna 416 and each receiving antenna 418, will dictate the nature of the out-of-plane propagation. The transmission and reception of microwave-frequency RF signals may also take place with antennas in the same array of antennas, such that data collection is in a transverse plane (e.g., one of transverse planes P₁ or P₂), as is done by assembly 100 of FIGS. 2 and 3. Thus, the combination of data acquisition in selectable transverse planes and out-of-plane configurations, provides true 3-D data gathering of the microwave-frequency RF signals propagated through and/or around biological tissue 422 (i.e., in vivo breast tissue). An optical scanner (not shown) may be positioned to image the in vivo tissue within illumination tank 404, as done by optical scanner 139 of FIG. 3, for spatially co-registering reconstructed image data of the microwave-frequency RF signals with a 3-D rendering of object surface.

Dividing antennas into first and second arrays 402, 403 reduces the data acquisition times associated with collecting measurements for 3-D microwave tomographic imaging because the number of possible vertical antenna position permutations for signal detection is decreased compared to the case where a single actuator controlled each antenna. Additionally, the alternative of acquiring sufficient 3-D data using a fixed 3-D antenna array for microwave imaging requires a very large number of antennas, which significantly increases the expense of the assembly because of the associated complex circuitry that would be necessary.

One exemplary arrangement for assembly 400 provides a pair of second actuators 417 each having a drive shaft 439 for vertically moving second mounting platform 437 mounted therewith. Second array of antennas 403 are mounted upon second mounting platform 437, and surround a hole 470 through which a drive shaft 438, vertically movable by first actuator 414, extends. A first mounting platform 436 is mounted with drive shaft 438 and has first array of antennas 402 mounted thereon. First mounting platform 436 overlaps second mounting platform 437 vertically over second array of antennas 403 and has an array of holes 405 extending therethrough and disposed between first array of antennas 402. Holes 405 are configured such that second array of antennas 403 may be extended through first mounting platform to form first and second antenna arrays 402, 403 into an interleaved, circular group of antennas. Alternatively, another arrangement for first and second actuators 414, 417 of assembly 400 may include actuator 414 connected to first mounting platform 436 through drive shaft 438 and a single second actuator 417 centrally positioned on top of first mounting platform 436 and connected to second mounting platform through drive shaft 439.

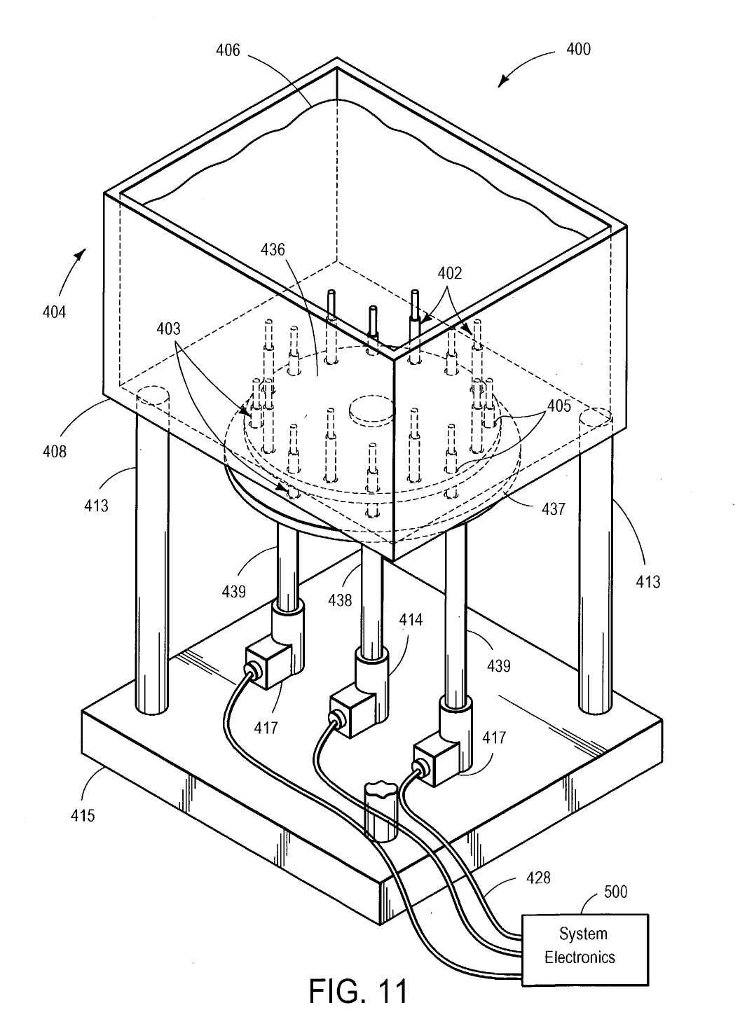

FIG. 11 shows how first and second antenna arrays 402, 403 extend through an illumination tank base 408 into a liquid coupling medium 406 within illumination tank 404. Seals (e.g., seals 132 of FIG. 3) are positioned within bores 434 formed into illumination tank base 408 through which the antennas 402, 403 extend. Similar to assembly 100 of FIGS. 2 and 3, legs 413 may support illumination tank 404 above base support 415.

A schematic illustration of a patient 472 undergoing a microwave imaging procedure is shown in FIG. 12. Patient 472 lies prone on a support table with breast tissue 422 as the particular in vivo biological tissue that is to be imaged pendant in liquid coupling medium 406 of illumination tank 404. First and second actuators 414, 417 then selectively vertically position first and second antenna arrays 402, 403, respectively, to surround differing portions of breast tissue 422. Microwave-frequency RF signals may then be transmitted by transmitting antenna 416 and received by any number of receiving antennas 418 in either or both of the first and second antenna arrays 402, 403, depending on the particular microwave imaging scheme. Transmitting antenna 416 may, of course, be located on either of the antenna arrays 402, 403. Alternatively, illumination tank assembly 400 could be configured in a similar fashion to assembly 200 of FIGS. 7 and 8, where drive shafts 438, 439 extend through bores 434 into illumination tank 404 such that antennas arrays 402, 403 and mounting platforms 436, 437 are positioned fully within tank 404.

When utilizing system 10 of FIG. 1, assembly 100 of FIGS. 2 and 3, assembly 200 of FIGS. 7 and 8, assembly 300 of FIG. 9, and assembly 400 of FIGS. 10 and 11, as medical microwave imaging data acquisition systems, a permittivity-compatible liquid coupling medium is desired. Improved microwave imaging of the electrical properties (e.g., conductivity and permittivity) for certain types of in vivo biological tissue, in one example, human breast tissue, are realized by the addition of glycerol to water, or glycerol to a saline solution, to form liquid coupling mediums 106, 206, 306 and 406 of microwave imaging systems 100, 200, 300 and 400, respectively. Glycerol may be referred to as “glycerine” herein, and the glycerine/water or glycerine/saline mixtures may be referred to generally as “glycerine mixtures”. Reduction of the contrast between the particular liquid coupling medium and the imaged object, achieved by the glycerine mixtures, is one method for improving imaging performance. The low permittivity characteristics of the glycerine mixtures may provide the benefits of: (a) reduction of 3-D wave propagation image artifacts when imaging schemes assume a 2-D model, (b) reduction of the effective imaging slice thickness when imaging in a transverse plane through the imaged object, (c) improvement in property characterization for large, low permittivity scatters, and (d) improved inclusion detection within the imaged object and artifact reduction. 3-D wave propagation image artifacts are typically more problematic when using a relatively large diameter array of antennas, and when lower frequency microwaves are used for imaging; however, the glycerine mixtures minimize the effects regardless of array diameter and frequencies of microwave transmission.

To better understand the concept of the effective imaging slice thickness when conducting microwave imaging, a sphere is examined as an exemplary object to be imaged, as taught in “Quantification of 3D field effects during 2D microwave imaging,” by P. M. Meaney, K. D. Paulsen, S. Geimer, S. Haider, and M. W. Fanning, IEEE Transactions on Biomedical Engineering, Volume 49, 708-720 (2002), incorporated herein by reference. For a single image slice through an ideal sphere, the recovered object diameter and the material property are important parameters for determining the effective imaging slice thickness. The effective diameter can be computed for each image by first integrating the recovered property parameter (e.g., conductivity or permittivity value)—with the exact background value subtracted from it—over the region of interest surrounding the object location and then comparing it with the result for the same integration over the exact distribution for a circular object with the known electrical properties; the effective diameter of the recovered object being the diameter of the circle for the exact solution required to make these two quantities equal. In general, 8 cm diameter integration domains centered on the recovered objects may be chosen for these computations, which are sufficient to capture their smoothed presence in the reconstructed images.

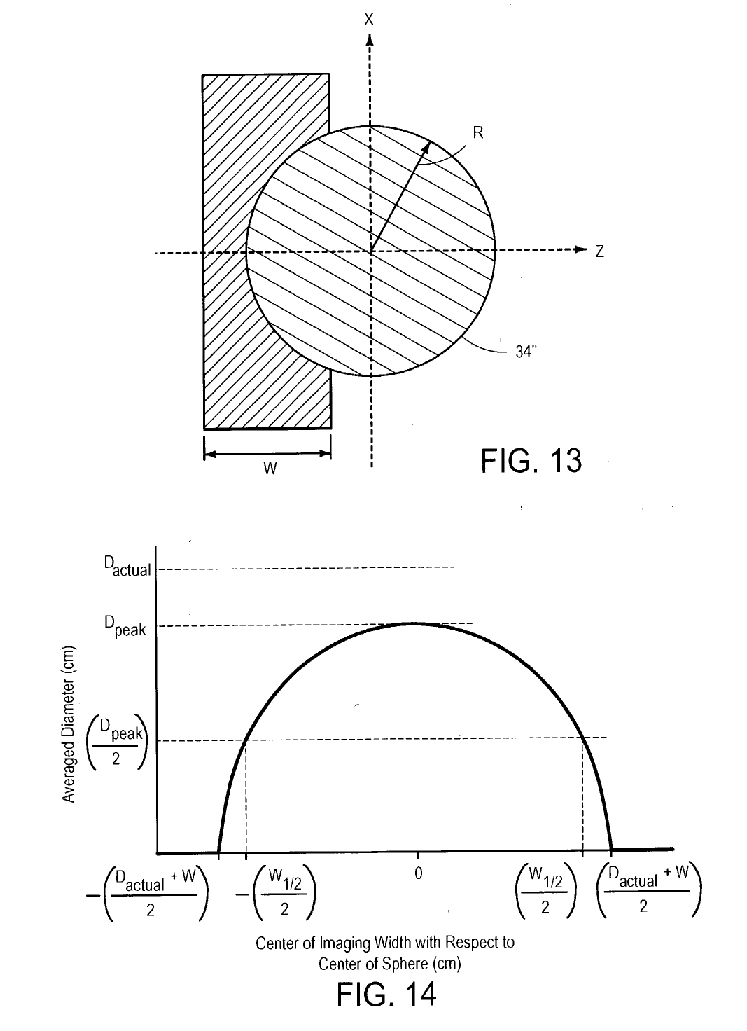

The effective imaging slice thickness, or averaging width, may then be estimated from phantom sphere experiments. FIG. 13 shows a diagram of the slice averaging width used in analyzing recovered object effective diameters. The averaging width is the vertical distance, or Z-coordinate (i.e., along the longitudinal axis L in FIG. 2) over which the horizontal diameters of an imaged object (e.g., biological tissue 34′, FIG. 4) are averaged. The averaging width is calculated for a sphere 34″ as the imaged object in these experiments. Each sphere 34″ is individually raised through the effective imaging plane with measurement data acquired at set vertical intervals. The effective diameters for each sphere 34″ may be calculated at each of these positions, preferably with the permittivity images as the electrical property image when a high contrast background such as saline is used. These permittivity images are generally more consistent than the respective conductivity images in the high background contrast situations, and plotted as a function of vertical position. In the cases where a relatively low contrast background is employed, both the permittivity and conductivity images may be used to compute the effective diameters. It is worth noting that for the cases where the conductivity images are inconsistent, especially for high contrast situations, this inconsistent conductivity image is one form of a 3D artifact. A more common example of a 3D artifact is when an object appears in either the permittivity or conductivity images that is physically not located within the 2D plane transected by the antenna array (e.g., antenna array 402, FIGS. 10-12), but for which there is a corresponding object physically present above or below that imaging plane at essentially the same X and Y coordinates.

Two metrics were derived from the calculated effective diameters. First, the recovered sphere half width (W_{1/2}), is the physical distance the averaging width is moved from one side of sphere 34″ at a point where the effective diameter is ½ its peak to the corresponding ½ peak point on the opposite side of sphere 34″. Previous experimentation has indicated that W_{1/2} is generally an over estimate of the averaging width. The second quantity, illustrated in FIG. 14, involves comparing the peak value of the effective diameter curve (Dₚₑₐₖ) with the actual sphere diameter (D_{actual}). More specifically, FIG. 14 shows a plot of the averaged diameter as a function of the averaging width center with respect to the sphere center (shown in FIG. 13) at the intersection of the X-axis and the Z-axis. If the diameters of the actual sphere are ideally averaged over a finite width (W) and plotted as a function of the center of the averaging span with respect to the sphere center, as shown in FIG. 14, Dₚₑₐₖ is less than D_{actual}. In fact, as the size of the averaging width increases, Dₚₑₐₖ decreases. The values Dₚₑₐₖ and D_{actual} can be used to estimate the averaging width, W. For cases where W is greater than D_{actual}, Dₚₑₐₖ can be expressed in terms of W and D_{actual} as:

$${D}_{peak} = \frac{π {D}_{actual}^{2}}{4 W} \qquad ( 1 )$$

or for cases where W is less than D_{actual}, Dₚₑₐₖ can be expressed as:

$${D}_{peak} = \frac{1}{2} \sqrt{{D}_{actual}^{2} - {W}^{2}} + \frac{{D}_{actual}^{2}}{2 W} arcsin ( \frac{W}{{D}_{actual}} ) \qquad ( 2 )$$

From either Eq. (1) or Eq. (2), whichever is appropriate, an averaging width W can be computed based on Dₚₑₐₖ and D_{actual}. Therefore, the overall effective imaging slice thickness may be determined from the average of W and W_{1/2}. Although Eq. (1) and (2) are applicable only for spheres as the imaged objects, comparable equations may be derived using the same methodology for other known geometric shapes.

The permittivity values for typical human breast tissue range from about 10 to 20 for frequency bands of about 300 MHZ to 3000 MHZ. However, the specific permittivity value varies within each breast because the breast is composed of both adipose tissue (essentially fat) and fibroglandular tissue, with each containing varying amounts of water. Because water has a fairly high relative permittivity value, around 75 to 80 for frequency bands of about 300 MHZ to 3000 MHZ, the differing water content for each kind of breast tissue causes the permittivity to vary across the breast. Additionally, there may be significant variation in breast tissue composition from patient to patient, giving further variability to breast permittivity values. Glycerine mixtures provide a liquid coupling medium with electrical properties that can be adjusted to have low contrast with breast tissue of a specific patient. Because of the miscibility with water, the permittivity of a glycerine mixture may be adjusted by further dilution with water. Further, to a limited degree additional sodium chloride can be added to a glycerine mixture to somewhat independently select a particular conductivity value for the liquid coupling medium.

Glycerine's chemical formula is CH₂OH—CHOH—CH₂OH where the hydroxyl groups (OH) attached to each carbon atom facilitate mixing with water. Glycerine is not harmful when in contact with human skin, and may be, in rare instances, a slight irritant in concentrations of over 95%. Glycerine also provides the advantages of being innocuous to the environment and bacteria “neutral”, or in other words, bacteria essentially cannot grow within a glycerine sample. This allows a quantity of glycerine used as a component of a liquid coupling medium to be reused from patient to patient, especially if sterilized between patient exams.

In addition to using the aforementioned glycerine mixtures, other polyols containing additional carbon and hydroxyl groups may used in a mixture with water or saline as a permittivity-compatible liquid coupling medium for microwave imaging. Such polyols have the following chemical formula: CH₂OH—(CHOH)ₙ—CH₂OH, wherein n may have a value ranging from 1 (in the case of glycerine) to 10, for a total of 3 to 12 carbon groups. The particular polyol chosen should be, at most, only a mild irritant to human skin, and essentially non-harmful upon contact therewith. Additionally, the chosen polyol is ideally innocuous to the environment and bacteria “neutral”.

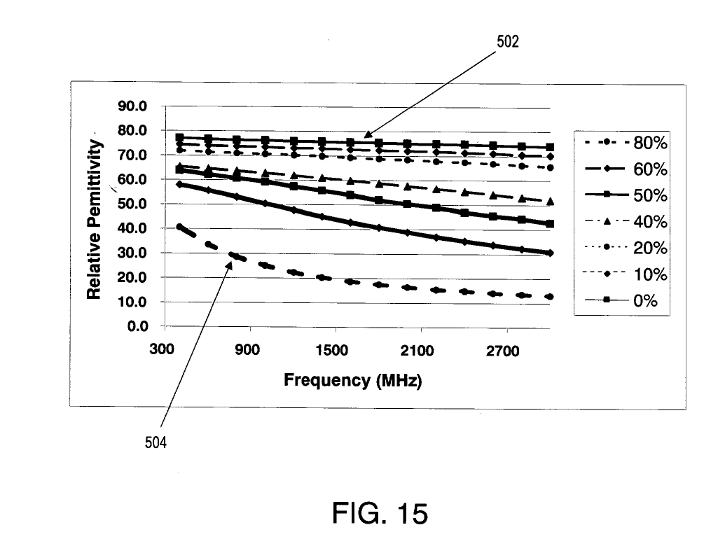

FIG. 15 shows a graph of the relative permittivity versus frequency for a range of mixture ratios of glycerine and water. The top curve 502 represents water. The bottom curve 504 represents mixtures of glycerine and water having 80 percent by volume of glycerine. The curves in between the top curve and the bottom curve represent glycerine/water mixtures of varying percentages of glycerine from about 10% to about 60%, as indicated. Each curve of the glycerine/water mixtures exhibits consistent dispersion characteristics—that is, the relative permittivity decreases monotonically for all frequencies as a function of increased glycerine percentage (X_{Glyc}) in the glycerine/water mixtures, with the slope generally being the steepest at the lower frequencies. What is important to note is that the permittivity decreases with respect to the increase in percent glycerine content in the mixtures. Thus, by knowing or approximating the tissue composition of a breast, the glycerine to water ratio of the liquid coupling medium can be tailored to the specific breast type.

In the microwave imaging systems 100, 200, 300, 400, the antenna arrays operate more optimally in a lossy liquid coupling medium (i.e., the antennas have an acceptable return loss—10 dB or better over a wide bandwidth of around 300 to 3000 MHz), the term lossy refers to attenuation of a microwave-frequency RF signal that would be observed if a plane wave were propagating through the medium. The lossiness of the liquid coupling medium effectively acts as a resistive load to the antennas, which are, generally, resonant structures operating over relatively narrow bandwidths with an overall size being related to the resonant frequencies of the antennas.

Although it is desired to have a liquid coupling medium that is somewhat lossy, the medium may have an optimal range of conductivity in order for the microwave antenna signals to propagate sufficiently through the medium to the imaged object (e.g., breast tissue) and to the other antennas, while also preserving the broadband characteristics of the antennas. Increasing the conductivity of the glycerine mixture allows for increasing the operating bandwidth of an array of antennas in the liquid coupling medium to a full frequency decade by resistively loading the antennas. Water and glycerine by themselves have quite low loss, although water's conductivity will increase monotonically with frequency. The unusual characteristics of water and glycerine mixtures thus provide a favorable behavior of conductivity.

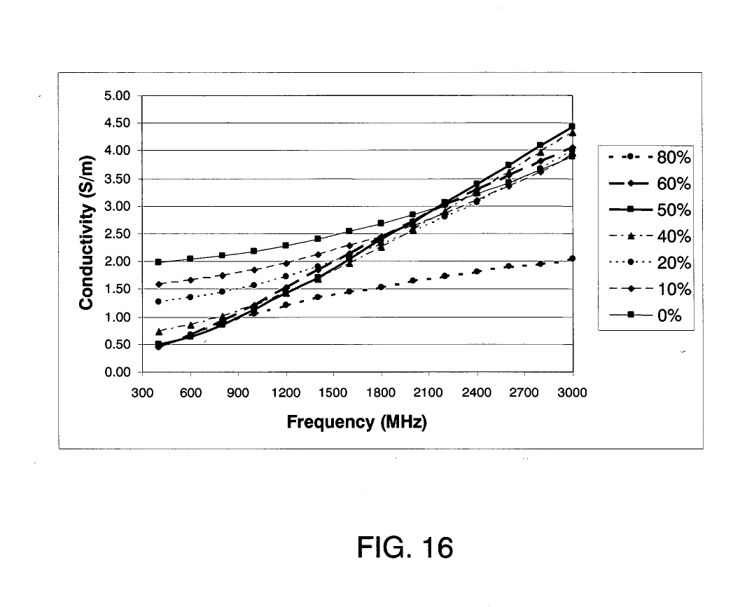

FIG. 16 shows graphs of conductivity versus frequency for various mixture ratios of water and glycerine. In the range of X_{glyc}=40% to 60%, there is a very steady increase in conductivity with frequency. Beyond about 60%, the conductivity at the higher frequencies starts to drop such that by about 80% glycerine at 3000 MHz, the conductivity only reaches about 2.0 S/m. By about 87% (not shown in the graph), the conductivity is relatively flat across the frequency band of 1000 MHz to 3000 MHz with a constant value of roughly 1.0 S/m. Above 87%, the whole curve drops quickly to the nominal conductivity value of 100% Glycerine, which has a low conductivity across the operating bandwidth of around 300 to 3000 MHz. A conductivity of 0.6 Mhos per meter (S/m) is roughly the lower limit for suitable resistive loading of the antenna arrays over a frequency range of about 300 to 3000 MHz.

It should be noted that the antennas do operate quite well—with respect to their return loss—even for the glycerine concentrations where the conductivity approaches values as high as 4.0 S/m at the higher end of the frequency range (i.e., near 3000 MHz). However, at the high conductivity values at these higher frequencies, it is difficult to propagate a microwave-frequency RF signal all the way across the imaging zone (i.e., the region within the array of antennas) which, in one example, is a transverse plane having a diameter of about 15 cm. Therefore, the resistive loading requirement for the antennas may be balanced with keeping the liquid coupling medium conductivity as low as possible for signal propagation across the imaging zone. To facilitate detection of highly attenuated signals, the antenna arrays and associated system electronics receiving the microwave signals may be configured to have a dynamic range of 130 dB. It has been found that liquid coupling medium mixtures on the order of about X_{Glyc}=70% to X_{Glyc}=90% provide a good balance in terms of resistive loading without excessive signal attenuation, while also providing permittivities in the range of what is observed for most human breast tissue, with X_{Glyc}=87% being one exemplary ratio across a fairly broad range of breast tissue properties. However, there may be a limitation in how much the permittivity can be reduced since above about X_{Glyc}=90%, the conductivity decreases to levels below 0.6 S/m across the frequency bands of interest and essentially does not independently increase through the addition of NaCl.

Utilization of the glycerine mixtures as a low-permittivity liquid coupling medium has also facilitated the use of the log-magnitude/phase Gauss-Newton iterative image reconstruction regularization algorithm described in the aforementioned “Microwave image reconstruction utilizing log-magnitude and unwrapped phase to improve high-contrast object recovery” by P. M. Meaney, K. D. Paulsen, B. W. Pogue, and M. I. Miga, IEEE Trans. MI, Volume MI-20, 104-116 (2001), along with improved imaging quality when the reconstruction parameter mesh is conformed to the actual target geometry. The former types of image reconstruction algorithms are especially useful when dealing with scattered electric field phase changes that exceed −180° to +180°, or −π to +π radians, where information may be lost due to phase wrapping. In the case of microwave imaging of breast tissue, scattered phases have been observed as high as 5π depending on the cross-section size of the imaged breast, operating frequency of the microwave imaging system, and the particular liquid coupling medium. Particularly, if there is contrast between the liquid coupling medium and the breast tissue, the scattered phase changes generally increase in magnitude with the operating frequency. While image reconstruction could take place at lower frequencies (e.g., 300 to 700 MHz), performing microwave imaging at higher frequencies is also desired because the spatial resolution is proportional to the wavelength associated with the operating frequency. It has been demonstrated that the utilization of data collected over a wide frequency range allows for unwrapping of the phases of the measured data.

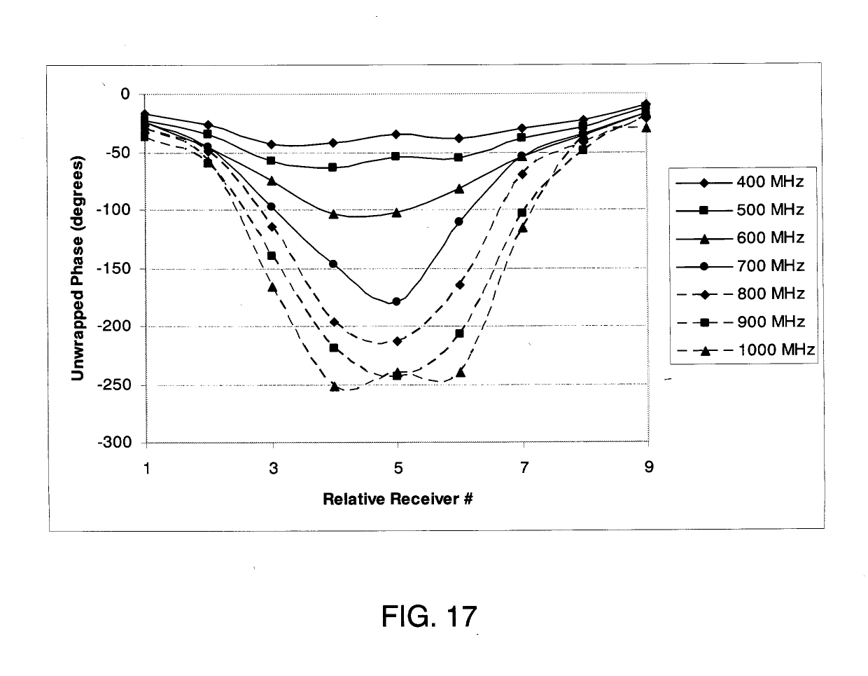

FIG. 17 shows a graph of the unwrapped phases for a range of frequencies measured at nine antenna receivers due to a single monopole microwave antenna transmitting microwave-frequency RF signals across one transverse imaging plane through a scattered density human breast pendant in a 70:30 (X_{Glyc}=70%) glycerine/water liquid coupling medium. In a first order observation, it appears that as the operating frequency increases, the object (i.e., breast) projection becomes more refined with the steep gradients to either side of the curves clearly delineating the object's size and position. At the lowest frequency, 400 MHz, the phases can be readily unwrapped by comparing the phases at adjacent receiver positions, based on a criteria that the phase difference between two adjacent receiving antennas should not exceed 180°. However, this is often not possible at higher frequencies since the difference in phases measured at adjacent receiving antennas can easily exceed 180°. Thus, at higher frequencies, instead of comparing the phases at adjacent receiver positions, data is collected at each antenna for the full frequency range at small frequency intervals and the phases are compared with values for the same antenna but at adjacent frequencies defined by the frequency interval chosen. Utilizing suitably small frequency intervals, it can be verified that the corresponding scattered field phases should vary only slightly between adjacent frequencies. Using the 400 MHz data curve as an unwrapped baseline, the data for all of the higher frequencies can readily be unwrapped.

In addition to using glycerine/water and glycerine/saline mixtures as suitable liquid coupling mediums, water, glycerine, an oil and an emulsifier may be combined into a mixture for use as a liquid coupling medium. The larger the amount of oil added to the mixture, the more emulsifier is needed to facilitate the water and oil mixing together, along with the glycerine, to form a substantially homogeneous mixture. The oil can be any oil that is not harmful when contacted by human skin, and when combined with the glycerine and water, achieves the desired electrical properties for a liquid coupling medium described herein. The glycerine helps maintain the necessary low permittivity characteristic of the oil mixture. It should also be understood that the aforementioned polyols may be substituted for glycerine in the oil mixture.

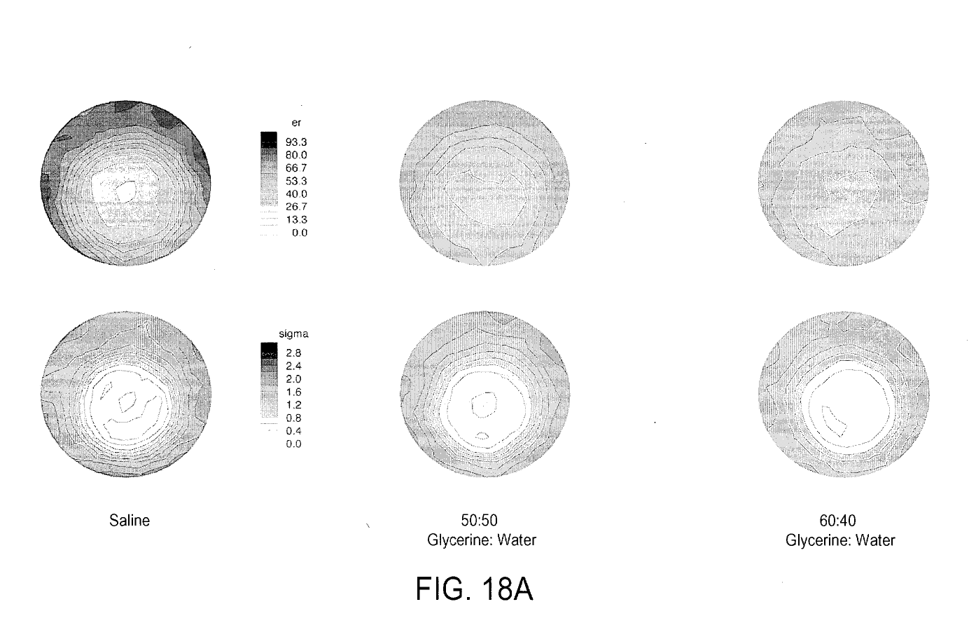

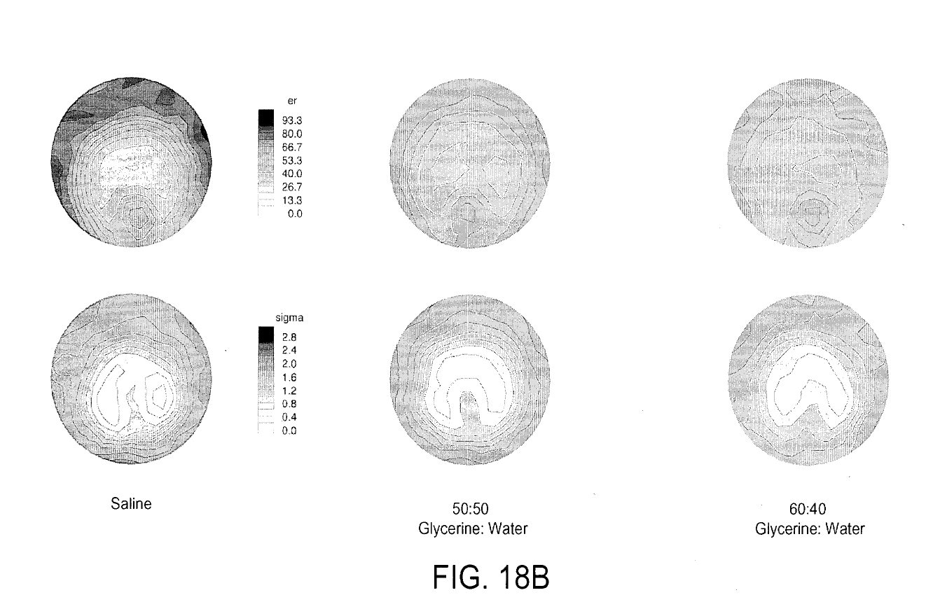

FIGS. 18A-20C show image reconstructions generated with microwave imaging system 10. The images were reconstructed using a Gauss-Newton iterative algorithm with a Marquardt regularization scheme. Sixteen forward solutions at each iteration were computed utilizing a hybrid of a finite element method for representing a heterogeneous imaging zone and a boundary element method for representing a homogeneous background region. The 15 cm diameter array of 16 monopole antennas surrounded the 13 cm diameter imaging zone, which was discretized into 2012 nodes and 3878 finite elements and surpassed criteria of 10 samples per wavelength and 7 samples per exponential decay at the operating frequency of 900 MHz. Each of the 16 antennas operates in both the transmit and receive mode with measurement data being recorded only at 9 of the antenna sites opposite each transmitting antenna for this example for a total of 144 observations per experiment. The images of FIGS. 18A-20C were reconstructed on a much more coarse parameter mesh having 142 nodes and 246 elements to minimize the size of the reconstruction problem. In addition, calculation of the electrical property updates was performed using the log-magnitude/phase Gauss-Newton iterative image reconstruction algorithm which is particularly well-suited for imaging large, high-contrast objects where wrapping of the measured field phases may be a problem. Also, an adjoint procedure has been applied to reduce the time to calculate the Jacobian matrix used in computing the electrical property updates at each iteration. This procedure has yielded the reconstruction of a single image with ten iterations in less than one minute.

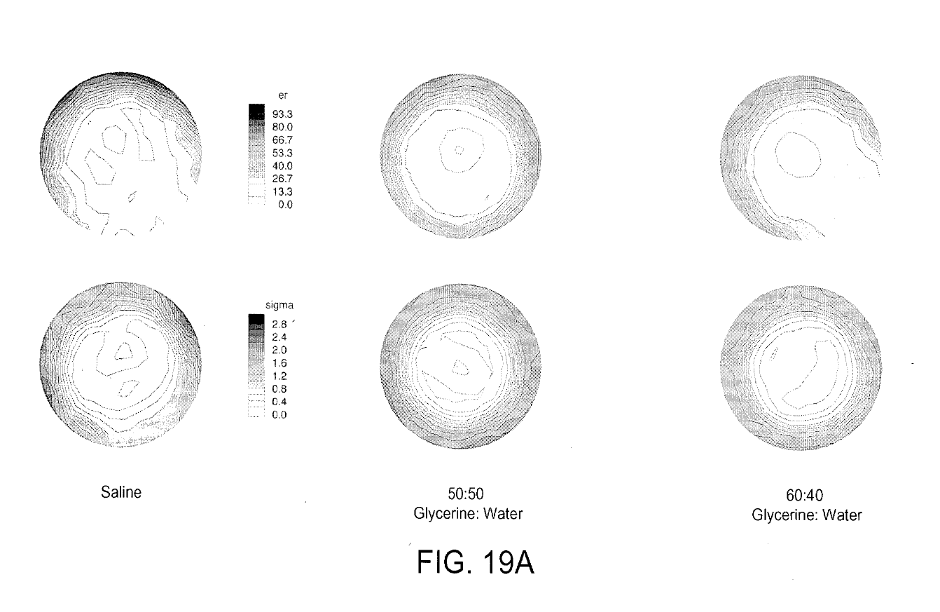

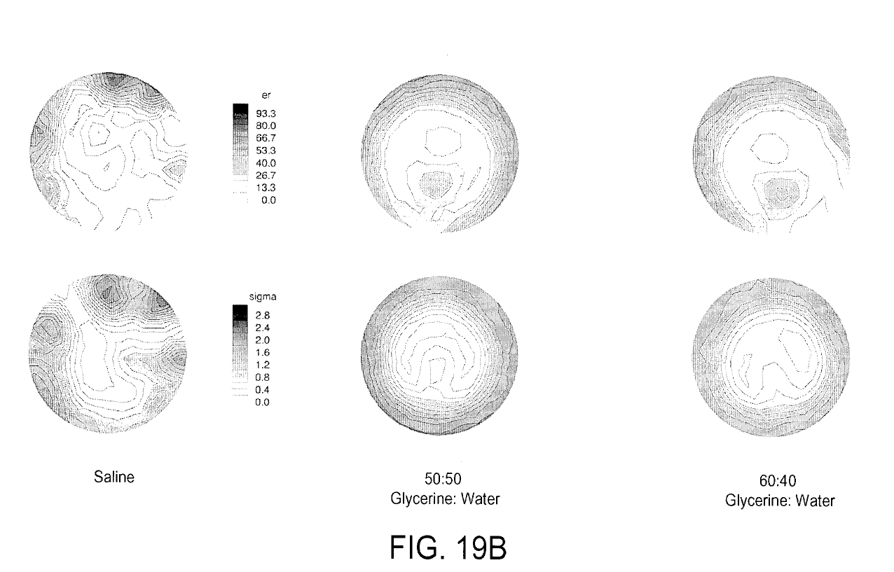

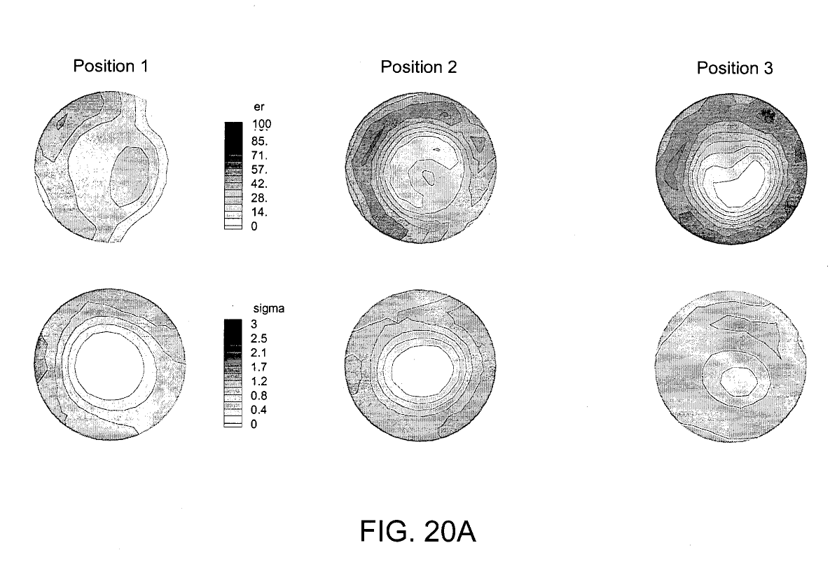

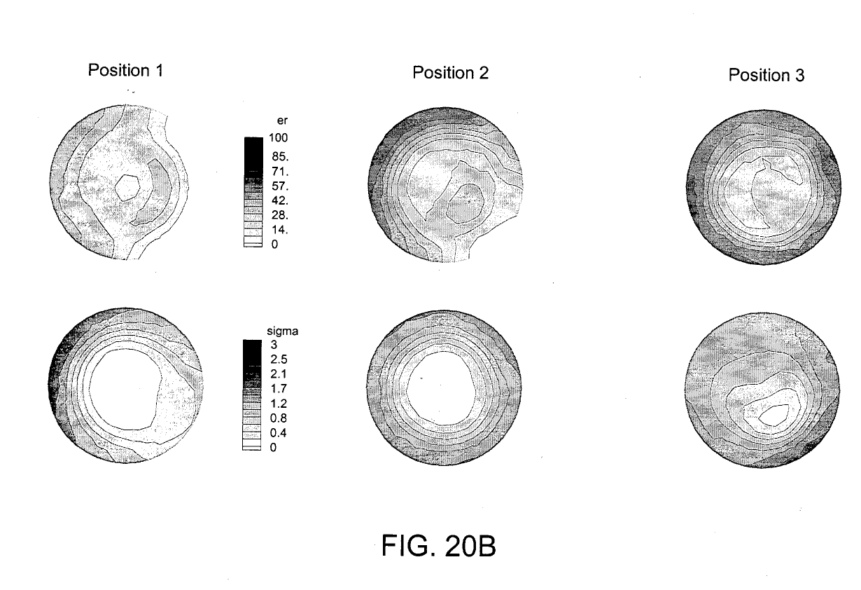

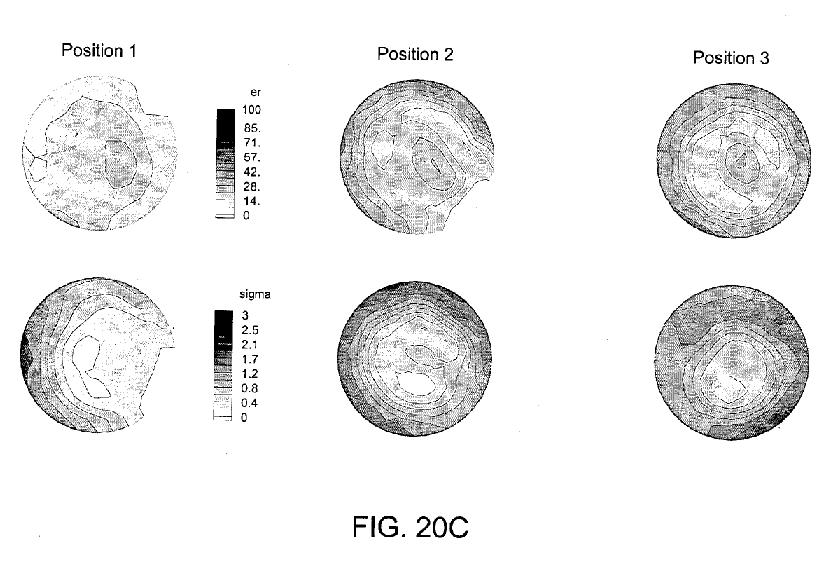

The image reconstruction and analysis of the resultant images is performed to assess the improvement in the electrical property recovery for large imaging targets as a function of the reduced contrast between the imaged object and the background liquid coupling medium. Reconstructed images of low-dielectric cylinders with and without small inclusions from measurement data are shown in FIGS. 18A, 18B, 19A and 19B. These results are used to assess the accuracy of electrical property recovery and the detectability of a localized heterogeneity. Similarly, reconstructed images of human breast tissue from measurement data are shown in FIGS. 20A-20C. In each of FIGS. 18A-20C, the top row of images represent permittivity images and the bottom row of images represent conductivity images.

### EXAMPLE 1

Microwave imaging experiments were performed at 900 MHz with the array of monopole antennas positioned in the illumination tank containing 0.9% saline, X_{Glyc}=50% and X_{Glyc}=60% mixtures for the liquid coupling medium, having electrical properties of (1) εᵣ=77.1, σ=1.72 S/m, (2) εᵣ=55.9, σ=1.64 S/m, and (3) εᵣ=47.9, σ=1.35 S/m, respectively, to provide a broad range of background permittivities. In these experiments, the transverse imaging plane was positioned 7 cm below the surface of medium 106 to minimize effects of signal reflections at the air/liquid interface. An 8.7 cm diameter agar gel cylinder, having a conductivity of σ=0.60 S/m and a permittivity of εᵣ=29.3 at 900 MHz, was imaged to examine the effects of background contrast with the recovery of property distributions for large imaging targets. The agar gel cylinder was imaged both as a homogeneous phantom and with an offset 1.9 cm diameter saline inclusion. In all cases the initial estimate for image reconstruction included a centrally located, rough-edged 9.1 cm diameter circle having εᵣ=26.5 and σ=0.56 S/m for the agar cylinder phantom surrounded by the known background medium. For the cases without the inclusion, seen in FIG. 18A, the permittivity images are generally quite uniform across the cylinder diameter with the recovered values only slightly below the background for the X_{Glyc}=60% case. The conductivity images are also quite uniform across the cylinder diameter; however, there is a modest artifact in the center of the recovered object for the saline and X_{Glyc}=50% cases where the conductivity exhibits a small increase. It is interesting to note that for all three backgrounds the object shape and position, as well as electrical properties, are accurately characterized.

The images for the agar cylinders with the 1.9 cm diameter saline inclusions, seen in FIG. 18B, generally characterize the object's shape, location and electrical properties. In all three background media, the permittivity image components detect and localize the inclusion quite well; however, there is significantly more variation in the recovered inclusion conductivity. In these images, the inclusion is visible as an increased conductivity indentation in the recovered object perimeter. However, with saline as the liquid coupling medium, the conductivity indentation extends further across the object, which alters considerably the nature of the reconstruction relative to the actual phantom.

### EXAMPLE 2

Microwave imaging experiments were also performed with a 10.7 cm diameter cylinder of molasses having a conductivity of σ=0.36 S/m and a relative permittivity of εᵣ=16.0 at 900 MHz. For FIGS. 19A and 19B, an array of monopole antennas was positioned in the illumination tank containing 0.9% saline, X_{Glyc}=50% and X_{Glyc}=60% mixtures for the liquid coupling medium, having electrical properties of (1) εᵣ=77.1, σ=1.72 S/m, (2) εᵣ=55.9, σ=1.64 S/m, and (3) εᵣ=47.9, σ=1.35 S/m, respectively, to provide a broad range of background permittivities. Similar to Example 1 where the agar gel cylinder was used, the molasses cylinder was imaged both as a homogeneous phantom and with an offset 1.9 cm diameter saline inclusion. In all cases, the initial estimate for image reconstruction included a centrally located, rough-edged 9.1 cm diameter circle having εᵣ=16.0 and σ=0.36 S/m surrounded by the known background medium. The initial estimate is provided to ensure that the results are not biased by inappropriate starting points to the iterative reconstruction process.

For the phantoms without inclusions, the overall property values are recovered quite well; however, there are more artifacts in the saline background case—primarily incorrect permittivity and conductivity increases contrast in the upper quadrant of the object. These artifacts generally decrease as a function of decreasing permittivity between the molasses cylinder and the background. In some images (in particular the permittivity images for the saline and X_{Glyc}=60% backgrounds) the object is smeared with the boundary of the imaging zone, which is most likely caused by positioning of the object too close to the edge of the imaging zone during the experiments. The images reconstructed for the inclusion cases are quite instructive. For the saline liquid coupling medium, the algorithm has converged to an uninteresting image. For the glycerine/water mixture mediums, the recovered images are quite accurate in terms of the cylinder properties, the inclusion size and its location. It appears that the X_{Glyc}=60% mixture recovers the properties of the saline inclusion better in the permittivity component, while the X_{Glyc}=50% mixture recovers the inclusion properties better in the conductivity component.

### EXAMPLE 3

Microwave imaging experiments were also performed with a human breast pendant in the liquid coupling medium formed of a X_{Glyc}=70% mixture and surrounded by the array of monopole antennas. The electrical properties of the liquid coupling medium at the three frequencies used are: (a) 500 MHz−εᵣ=47.4, σ=0.61 S/m, (b) 700 MHz−εᵣ=43.0, σ=0.88 S/m, and (c) 1000 MHz−εᵣ=37.4, σ=1.28 S/m. The degree of phase wrapping is substantially decreased in the X_{Glyc}=70% mixture having a low permittivity as compared to 0.9% saline or water alone as a coupling medium, especially at higher frequencies.

FIGS. 20A-20C show the recovered images at 500 MHz, 700 MHz and 1000 MHz, respectively, for three transverse imaging planes through the breast relatively close to a chest wall of a patient. Position 1 is for the plane closest to the chestwall with each subsequent position corresponding to planes 1 cm and 2 cm away from position 1. The images of FIGS. 20A-20C for each plane are in relatively permittivity and conductivity pairs (permittivity images positioned above the corresponding conductivity images). In general, there is a ring of higher permittivity and conductivity near a portion of the perimeter of each image associated with the higher property-valued liquid coupling medium. Consistent bands of low permittivity around another portion of the image perimeter are present when the algorithm attempts to compensate for the fact that low permittivity tissue extends outside of the imaging zone where the algorithm assumed only the higher permittivity liquid coupling medium is present. Additionally, the size of the breast cross-sections uniformly decrease as the distance from the chest wall increases, as expected.

For most of the permittivity images, there appears to be a small region within each recovered object where the permittivity is noticeably higher than for the surrounding tissue, with the exception being the 500 MHz, position 3 case where presumably the resolution is not sufficient to extract this feature and instead presents a higher permittivity smooth indentation into the object. The difference in permittivities for the two zones may be the result of uneven distributions of fatty and fibroglandular tissue with the fattier (lower permittivity) sections concentrating nearer the breast perimeter. It does appear that the sizes of the recovered shapes, along with the definition of the internal higher permittivity and conductivity structures, appear to increase with operating frequency. These differences are most likely 3-D artifacts which are reduced with increased operating frequency.

The information gathered in the first two examples shows that the electrical properties of large scatters, as well as inclusions, can be more accurately recovered when the contrast with the background is reduced. The information from the last example also shows that the 3-D wave propagation image artifacts of microwave imaging are reduced for water/glycerine and saline/glycerine background mixtures as compared to saline solutions or water alone. In terms of imaging of the breast, as the breast is pendant in our liquid-coupled imaging array, its shape is generally more conical than cylindrical which increase the chances that 3-D artifacts will be significant. Thus, the reduction of 3-D artifacts provides benefit. These improvements realized in the water/glycerine and saline/glycerine mixtures facilitate allowing for quantifiably distinguishing between fatty and fibroglandular tissue, as well as between benign and malignant tumors which will be a considerable asset in the clinical implementation of microwave breast imaging. The images produced over the frequency range of 500 MHz to 1000 MHz shown in FIGS. 20A-20C are rich in spectral information of the examined breast tissue with the higher frequency reconstructions producing images with the most detail about the internal structures of the breast.

We claim:

1. A low-contrast liquid coupling medium for microwave imaging of a sample, comprising:
a mixture of water and a polyol having three or more hydroxyl groups and 3 to 12 carbon atoms.

2. The liquid coupling medium of claim 1, the sample comprising biological tissue.

3. The liquid coupling medium of claim 1, the polyol comprising glycerol.

4. The liquid coupling medium of claim 3, wherein the mixture comprises a range from about 50 to 90 percent by volume of glycerol.

5. The liquid coupling-medium of claim 1, further comprising sodium chloride dissolved in the mixture.

6. The liquid coupling medium of claim 1, further comprising an emulsifier and an oil dissolved in the mixture.

7. In a microwave imaging apparatus having an illumination tank and an array of antennas, an improvement comprising:
seals integral with a base of the illumination tank through which the antennas slide into the illumination tank.

8. In a microwave imaging apparatus as in claim 7, a further improvement comprising:
a signal processor coupled to the antennas and configured for processing a demodulated signal representative of the microwave-frequency RF signal received by the one or more of the antennas, the signal processor being disposed outside of the illumination tank.

9. A system for imaging biological tissue with a microwave-frequency RF signal, comprising:
an illumination tank configured for accommodating the biological tissue, a plurality of bores formed into the tank, each bore having one or more seals formed therein;
a plurality of antennas extending through the bores and associated seals into the illumination tank, one or more of the plurality of antennas configured for receiving the microwave-frequency RF signal; and
a signal processor coupled to the receiving antennas and configured for processing a demodulated signal representative of the microwave-frequency RF signal received by the one or more of the plurality of antennas.

10. The system of claim 9, the signal processor comprising a plurality of receivers wherein each receiver is coupled to one of the receiving antennas and configured for generating the demodulated signal.

11. The system of claim 10, the signal processor comprising an A/D converter configured for generating a digital representation of the demodulated signal.

12. The system of claim 11, the signal processor coupled to one or more of the plurality of receivers and configured for comparing the phase and magnitude of the digital representations of the demodulated signal to that of a modulating waveform.

13. The system of claim 12, the signal processor further generating a visual representation of the conductivity and permittivity across the biological tissue from the comparison of the phase and magnitude of the digital representations of the demodulated signal to that of a modulating waveform.

14. The system of claim 9, the microwave-frequency RF signal having a frequency in a range of about 300 MHz to 3 GHz.

15. The system of claim 9, the demodulated signal comprising an intermediate frequency signal having a frequency in a range of about 1 KHz to 1 MHz.

16. The system of claim 9, the antennas being monopole antennas comprising:
a base region formed of a coaxial feed line having a center conductor, an insulator circumscribing the center conductor and a rigid outer conductor circumscribing the insulator;
a tip region extending out of the base and formed of the coaxial feed line without the rigid outer conductor; and
wherein the tip region extends to a first antenna end disposed within the illumination tank and the base region extends to a second antenna end disposed outside the illumination tank, the second antenna end having a connector formed therewith.

17. The system of claim 16, the connector configured for connection to a communications cable to carry signals between the signal processor and the monopole antennas.

18. The system of claim 9, further comprising an optical scanner for capturing optical image data of biological tissue disposed within the tank.

19. The system of claim 9, the plurality of bores being formed in a base of the illumination tank, the plurality of antennas extending along a longitudinal axis of the tank.

20. The system of claim 9, further comprising a transmitter configured for transmitting the microwave-frequency RF signal through one or more of the plurality of antennas such that the one or more antennas propagate the signal through the biological tissue.

21. The system of claim 20, wherein the plurality of antennas form a first array of antennas, and the transmission and reception of the microwave-frequency RF signal by the antennas is in a plane through the biological tissue transversely aligned with the illumination tank, the system further comprising a first actuator disposed outside of the illumination tank for adjusting the position of the first array of antennas within the illumination tank along a longitudinal axis of the tank, to select a particular transverse plane through the biological tissue.

22. The system of claim 21, further comprising a structural base onto which the illumination tank and the first actuator are positioned.

23. The system of claim 21, the first actuator comprising a computer-controlled linear actuator.

24. The system of claim 21, further comprising:
a mounting platform disposed outside of the illumination tank with which the first array of antennas are mounted; and
a drive shaft coupled to the mounting platform and selectively movable by the first actuator.

25. The system of claim 24, the first array of antennas having a threaded region for securing the antennas within a threaded bore of the mounting platform and a mounting flange disposed proximal to the threaded region for abutting the mounting platform.

26. The system of claim 20, the plurality of antennas forming a first array of antennas and a second array of antennas, the system further comprising one or more actuators disposed outside of the illumination tank, each actuator configured for adjusting the position of one of the first array of antennas and the second array of antennas within the illumination tank along a longitudinal axis of the tank, to selectively position the first array of antennas and the second array of antennas relative to one another;
wherein positioning of the first array of antennas and the second array of antennas provides three-dimensional data collection by one or more antennas of one of the first and second array of antennas detecting microwave signals transmitted by one or more antennas of the other of the first and second array of antennas, the first array of antennas and second array of antennas being positioned at differing transverse planes through the biological tissue.

27. The system of claim 26, wherein the one or more actuators includes a computer-controlled linear actuator.

28. The system of claim 26, further comprising:
a first mounting platform disposed outside of the illumination tank with which the first array of antennas are mounted;
a second mounting platform disposed outside of the illumination tank with which the second array of antennas are mounted; and
a drive shaft selectively movable by each actuator, each drive shaft coupled to one of the first mounting platform and the second mounting platform corresponding to the array of antennas movable by the actuator.

29. The system of claim 28, each antenna of the first array of antennas and the second array of antennas having a threaded region for securing the antenna within a threaded bore of the first mounting platform and the second mounting platform, respectively, and a mounting flange disposed proximal to the threaded region for abutting the respective mounting platform.

30. The system of claim 28, the first mounting platform having a plurality of bores formed therein and between the first array of antennas, the second mounting platform disposed at least partially beneath the first mounting platform to position the second array of antennas to extend through the bores to form the first array of antennas and second array of antennas in an interleaved, circular arrangement.

31. The system of claim 9, the seals comprising hydraulic seals.

32. The system of claim 9, the signal processor disposed outside of the illumination tank.

33. The system of claim 9, the illumination tank having a volume of a liquid coupling medium formed of a mixture of water and a polyol having three or more hydroxyl groups and 3 to 12 carbon atoms.

34. The system of claim 33, the polyol comprising glycerol, the mixture comprising a range from about 50 to 90 percent by volume of glycerol.

35. A system for imaging biological tissue with a microwave-frequency RF signal, comprising:
an illumination tank configured for accommodating the biological tissue, one or more bores formed into the tank and having one or more seals formed therein;
one or more drive shafts each extending through one bore and associated seals into the illumination tank;
one or more mounting platforms each mounted with the one or more drive shafts and disposed within the illumination tank;
a plurality of antennas mounted onto the one or more mounting platforms, one or more of the plurality of antennas configured for receiving the microwave-frequency RF signal; and
a signal processor coupled to the receiving antennas and configured for processing a demodulated signal representative of the microwave-frequency RF signal received by the one or more of the plurality of antennas.

36. The system of claim 35, the signal processor comprising a plurality of receivers wherein each receiver is coupled to one of the receiving antennas and configured for generating the demodulated signal.

37. The system of claim 36, the signal processor comprising an A/D converter configured for generating a digital representation of the demodulated signal.

38. The system of claim 37, the signal processor coupled to one or more of the plurality of receivers and configured for comparing the phase and magnitude of the digital representations of the demodulated signal to that of a modulating waveform.

39. The system of claim 38, the signal processor further generating a visual representation of the conductivity and permittivity across the biological tissue from the comparison of the phase and magnitude of the digital representations of the demodulated signal to that of a modulating waveform.

40. The system of claim 35, the microwave-frequency RF signal having a frequency in a range of about 300 MHz to 3 GHz.

41. The system of claim 35, the demodulated signal comprising an intermediate frequency signal having a frequency in a range of about 1 KHz to 1 MHz.

42. The system of claim 35, the antennas being monopole antennas comprising:
a base region formed of a coaxial feed line having a center conductor, an insulator circumscribing the center conductor and a rigid outer conductor circumscribing the insulator; and
a tip region extending out of the base and formed of the coaxial feed line without the rigid outer conductor;
wherein the tip region extends to a first antenna end disposed within the illumination tank and the base region extends to a second antenna end disposed outside the illumination tank, the second antenna end having a first connector formed therewith.

43. The system of claim 42, the first connector configured for connection to a communications cable to carry signals between the signal processor and the monopole antennas.

44. The system of claim 43, a cable bore formed through a wall of the illumination tank, the system further comprising:
a first communications cable section connected on a first end to the first connector and having a second connector formed on a second end of the cable section;
a coaxial connector bulkhead adapter having a first end connected to the second connector and a second end; and
a second communications cable section having a third connector formed at a first end and connected to the second end of the bulkhead adapter, the second communications cable extending to the signal processor.

45. The system of claim 35, further comprising a transmitter configured for transmitting the microwave-frequency RF signal through one or more of the plurality of antennas such that the one or more antennas propagate the signal through the biological tissue.

46. The system of claim 45, wherein the plurality of antennas form a first array of antennas mounted to one mounting platform mounted with one drive shaft, and the transmission and reception of the microwave-frequency RF signal by the antennas is in a plane through the biological tissue transversely aligned with the illumination tank, the system further comprising a first actuator disposed outside of the illumination tank for selectively moving the drive shaft along a longitudinal axis of the illumination tank to position the first array of antennas within the illumination tank to select a particular transverse plane through the biological tissue.

47. The system of claim 46, the first actuator comprising a computer-controlled linear actuator.

48. The system of claim 35, the first array of antennas having a threaded region for securing the antennas within a threaded bore of the first mounting platform and a mounting flange disposed proximal to the threaded region for abutting the mounting platform.

49. The system of claim 35, the plurality of antennas forming a first array of antennas mounted onto one mounting platform and a second array of antennas mounted onto another mounting platform, the system further comprising one or more actuators disposed outside of the illumination tank, each actuator configured for adjusting the position of one of the first array of antennas and the second array of antennas within the illumination tank along a longitudinal axis of the tank by moving the respective drive shafts, to selectively position the first array of antennas and the second array of antennas relative to one another;
wherein positioning of the first array of antennas and the second array of antennas provides three-dimensional data collection by one or more antennas of one of the first and second array of antennas detecting microwave signals transmitted by one or more antennas of the other of the first and second array of antennas, the first array of antennas and second array of antennas being positioned at differing transverse planes through the biological tissue.

50. The system of claim 49, wherein the one or more actuators includes a computer-controlled linear actuator.

51. The system of claim 49, each antenna of the first array of antennas and the second array of antennas having a threaded region for securing the antenna within a threaded bore of the respective mounting platform, and a mounting flange disposed proximal to the threaded region for abutting the respective mounting platform.

52. The system of claim 49, the mounting platform associated with the first array of antennas having a plurality of bores formed therein and between the first array of antennas, the mounting platform associated with the second array of antennas disposed at least partially beneath the first mounting platform to position the second array of antennas to extend through the bores to form the first array of antennas and second array of antennas in an interleaved, circular arrangement.

53. The system of claim 35, the one or more seals comprising hydraulic seals.

54. The system of claim 35, the signal processor disposed outside of the illumination tank.

55. The system of claim 35, the one or more bores formed in a base of the illumination tank, each drive shaft extending along a longitudinal axis of the tank.

56. The system of claim 35, the illumination tank having a volume of a liquid coupling medium formed of a mixture of water and a polyol having three or more hydroxyl groups and 3 to 12 carbon atoms.

57. The system of claim 56, the polyol comprising glycerol, the mixture comprising a range from about 50 to 90 percent by volume of glycerol.

58. A system for imaging biological tissue with a microwave-frequency RF signal, comprising:
an illumination tank configured for accommodating the biological tissue, one or more bores formed into the tank, each bore having one or more seals formed therein;
a support rod extending through each bore and associated seals into the illumination tank;
one or more antennas mounted with each support rod and disposed within the illumination tank; and
a signal processor coupled to the receiving antennas and configured for processing a demodulated signal representative of the microwave-frequency RF signal received by the one or more of the plurality of antennas.

59. The system of claim 58, further comprising a transmitter configured for transmitting the microwave-frequency RF signal through one or more of the antennas such that the one or more antennas propagate the signal through the biological tissue.

60. The system of claim 58, the one or more bores comprising a plurality of bores, the support rod extending through each bore forming a plurality of support rods, the system further comprising an actuator disposed outside of the illumination tank for adjusting the position of the plurality of support rods to selectively position the antennas along a longitudinal axis of the tank, to select a particular transverse imaging plane through the biological tissue.

61. The system of claim 60, each support rod mounted on a first end with a mounting platform disposed outside of the illumination tank and having one or more antennas mounted therewith on a second end, the mounting platform coupled to a drive shaft selectively movable by the actuator.

63. The system of claim 58, each antenna having a first connector formed therewith, the first connector configured for connection to a communications cable to carry signals between the signal processor and the antennas.

64. The system of claim 63, a cable bore formed through a wall of the illumination tank, the system further comprising:
a first communications cable section connected on a first end to the first connector and having a second connector formed on a second end of the cable section;
a coaxial connector bulkhead adapter having a first end connected to the second connector and a second end; and
a second communications cable section having a third connector formed at a first end and connected to the second end of the bulkhead adapter and extending to the signal processor.

65. The system of claim 58, the antennas comprising waveguide antennas.

66. A method of imaging biological tissue with a microwave-frequency RF signal, comprising the steps of:
positioning biological tissue within an illumination tank having a volume of a liquid coupling medium therein and having a plurality of antennas within the tank to surround the biological tissue;
generating a microwave-frequency RF signal;
transmitting the microwave-frequency RF signal through one or more of the plurality of antennas to propagate the signal through the biological tissue;
receiving the microwave-frequency RF signal propagated through the biological tissue; and
processing the received microwave-frequency RF signals to determine characteristics of the imaged biological tissue.

67. The method of claim 66, the steps of processing comprising:
generating a demodulated signal from the received microwave-frequency RF signal;
generating a digital representation of the demodulated signal;
comparing the phase and magnitude of the digital representations of the demoulated signals to that of a modulating waveform; and
reconstructing permittivity and conductivity images of the biological tissue using the phase and magnitude differences between the digital representation of the demodulated signal and the modulating waveform.

68. The method of claim 67, the demodulated signal comprising an intermediate frequency signal having a frequency in a range of about 1 KHz to 1 MHz.

69. The method of claim 68, further comprising:
optically scanning the biological tissue positioned within the illumination tank; and
generating a three-dimensional optical image of the biological tissue.

70. The method of claim 69, further comprising:
overlaying the conductivity and permittivity images with the three-dimensional optical image of the biological tissue to co-register features of the conductivity and permittivity images with the anatomy of the biological tissue.

71. The method of claim 66, the plurality of antennas forming a first array of antennas, the method further comprising displacing the first array of antennas along a longitudinal axis of the illumination tank for transmitting the microwave-frequency RF signal and receiving the microwave-frequency RF signal propagated through the biological tissue in selected transverse planes through the biological tissue.

72. The method of claim 66, the plurality of antennas forming a first array of antennas and a second array of antennas, the method further comprising displacing one or more of the first array of antennas and second array of antennas along a longitudinal axis of the illumination tank, and the step of transmitting the microwave-frequency RF signal comprises the transmission performed by one of the first array of antennas and the second array of antennas, and the step of receiving the microwave-frequency RF signal propagated through the biological tissue comprises the reception performed by the other of the first array of antennas and the second array of antennas, thereby facilitating detection of signals outside of a selected transverse plane of transmission defined by the transmitting array of antennas through the biological tissue.

73. The method of claim 72, the first array of antennas and the second array of antennas positioned in an interleaved, circular arrangement.

74. The method of claim 66, the liquid coupling medium comprising a mixture of water and a polyol having three or more hydroxyl groups and 3 to 12 carbon atoms.

75. The method of claim 74, the polyol comprising glycerol, the mixture comprising a volume of glycerol in a range from about 50 to 90 volume percent.

76. The method of claim 66, wherein the illumination tank has at least one sidewall and a base, the plurality of antennas extending into the illumination tank through seals integral with the base.

77. The method of claim 66, wherein the biological tissue is in vivo human breast tissue, the method being further for detecting malignant tissue.
